(19) United States
(12) Patent Application Publication
(10) Pub. No.: US 2014/0205566 A1
(43) Pub. Date: Jul. 24, 2014

(54) CYCLIC NUCLEUOSIDE DERIVATIVES AND USES THEREOF

(71) Applicants: Lv LIAO, Shanghai (CN); Fumiaki YOKOKAWA, Singapore (SG); Gang WANG, Singapore (SG)

(72) Inventors: Lv LIAO, Shanghai (CN); Fumiaki YOKOKAWA, Singapore (SG); Gang WANG, Singapore (SG)

(73) Assignee: NOVARTIS AG, Basel (CH)

(21) Appl. No.: 14/086,271

(22) Filed: Nov. 21, 2013

(30) Foreign Application Priority Data
Nov. 30, 2012 (SG) 201208891.0

(51) Int. Cl. C07H 19/20; A61K 45/06; A61K 31/7076

(52) U.S. Cl. CPC C07H 19/20; A61K 31/7076; A61K 45/06
USPC 424/85.4; 536/26.12; 514/48

(57) ABSTRACT

A compound of Formula (I) is provided that has been shown to be useful for treating a disease caused by a viral infection:

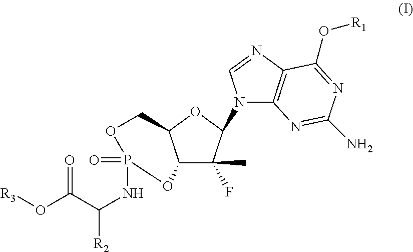

wherein R₁, R₂ and R₃ are as defined herein.

This application claims priority to Foreign Singapore Patent Application No. 201208891.0, filed 30 Nov. 2012, the contents of which are incorporated herein by reference in their entirety.

### FIELD OF THE INVENTION

The present invention relates to cyclic nucleoside derivatives, pharmaceutical compositions thereof, and their use for the prevention and treatment of viral infections, in particular viral infections caused by dengue virus. The present invention also relates to polymorphic forms of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate which are useful in the prevention and treatment of viral infections, in particular viral infections caused by dengue virus.

### BACKGROUND

Dengue fever is a febrile disease caused by one of the four dengue virus serotypes DEN-1, DEN-2, DEN-3 and DEN-4, which belong to the family Flaviviridae. The virus is transmitted to humans primarily by Aedes aegypti, a mosquito that feeds on humans.

Infections produce a range of clinical manifestations, from milder flu-like symptoms to the more severe and sometimes fatal hemorrhagic disease. Typical symptoms include fever, severe headache, muscle and joint pains and rashes. The more severe forms of the disease are dengue hemorrhagic fever (DHF) and dengue shock syndrome (DSS). According to the WHO, there are four major clinical manifestations of DHF: (1) high fever (2) haemorrhagic phenomena (3) thrombocytopaenia and (4) leakage of plasma. DSS is defined as DHF plus weak rapid pulse, and narrow pulse pressure or hypotension with cold, clammy skin and restlessness. The severity of DHF can be reduced with early detection and intervention, but subjects in shock are at high risk of death.

Dengue is endemic in tropical regions, particularly in Asia, Africa and Latin America, and an estimated 2.5 billion people live in areas where they are at risk of infection. There are around 40 million cases of dengue fever and several hundred thousand cases of DHF each year. In Singapore, an epidemic in 2005 resulted in more than 12000 cases of dengue fever.

Despite regular outbreaks, previously infected people remain susceptible to infection because there are four different serotypes of the dengue virus and infection with one of these serotypes provides immunity to only that serotype. It is believed that DHF is more likely to occur in subjects who have secondary dengue infections. Efficient treatments for dengue fever, DHF and DSS are being sought.

Yellow fever virus (YFV), West Nile virus (WNV), Japanese encephalitis virus (JEV), tick-borne encephalitis virus, Kunjin virus, Murray Valley encephalitis, St Louis encephalitis, Omsk hemorrhagic fever virus, bovine viral diarrhea virus, Zika virus and Hepatitis C virus (HCV) also belong to the family Flaviviridae.

WNV can be asymptomatic, or it can cause flu-like symptoms in some individuals. In some cases it causes neurological disorders, encephalitis, and in severe cases can result in death. WNV is also transmitted by mosquitoes. YFV is also transmitted by mosquitoes, and can cause severe symptoms in infected individuals. JEV is also transmitted by mosquitoes, and is either asymptomatic or causes flu-like symptoms, with some cases developing into encephalitis. The acute encephalitis stage of the disease is characterized by convulsions, neck stiffness and other symptoms. HCV is a blood-borne virus that is transmitted by blood-to-blood contact. In the initial (acute) stage of the disease, most subjects will not show any symptoms. Even during the chronic stage (i.e. where the disease persists for more than 6 months), severity of symptoms can vary from subject to subject. In the long term, some infected persons can progress to cirrhosis and liver cancer. The current treatment for HCV involves a combination of interferon alpha and ribavirin, an anti-viral drug. Efficient treatments for infections caused by these Flaviviridae viruses are being sought as well.

It has now surprisingly been found that cyclic nucleoside derivatives as presently disclosed are useful for the treatment of viral infections such as those caused by a virus of the family Flaviviridae, especially dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus, and other Flaviviridae viruses as described herein.

### SUMMARY OF THE INVENTION

The compounds described herein have been shown to be useful in the prevention and/or treatment of viral infections.

One aspect of the present invention provides compounds of Formula (I)

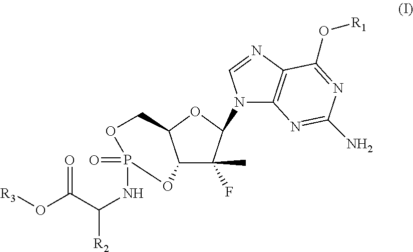

wherein

R₁ is methyl, ethyl, n-propyl or i-propyl;

R₂ is H, methyl, ethyl, n-propyl or i-propyl; and

R₃ is methyl, ethyl, n-propyl or i-propyl.

In one embodiment, the compound of Formula (I), has a structure of Formula (II)

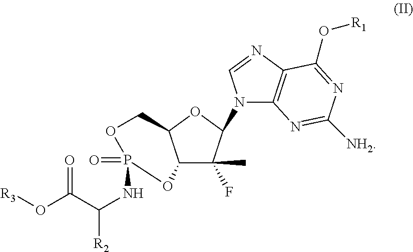

In another embodiment, the compound of Formula (I), has a structure of Formula (III)

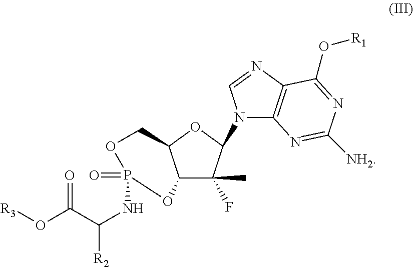

In yet another embodiment, the compound of Formula (I), has a structure of Formula (IV)

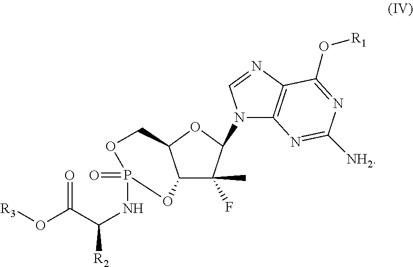

In still another embodiment, the compound of Formula (I), has a structure of Formula (V)

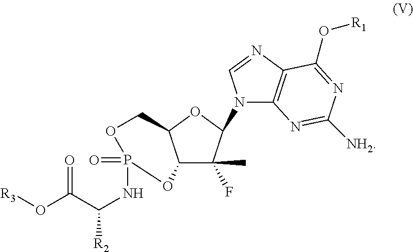

In another embodiment, the compound of Formula (I), is provided wherein R₁ is ethyl, n-propyl or i-propyl. In yet another embodiment, the compound of Formula (I), is provided wherein R₂ is H, methyl or i-propyl. In still another embodiment, the compound of Formula (I), is provided wherein R₃ is methyl, ethyl or i-propyl.

Representative compounds of Formula (I) include: (S)-methyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; (R)-isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; (S)-isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; isopropyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)acetate; isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)acetate; (S)-ethyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)-3-methylbutanoate; (S)-methyl 2-(((2R,4aR,6R,7R,7aR)-6-(2-amino-6-isopropoxy-9H-purin-9-yl)-7-fluoro-7-methyl-2-oxidotetrahydro-4H-furo[3,2-d][1,3,2]dioxaphosphinin-2-yl)amino)propanoate; and (S)-methyl 2-(((2R,4aR,6R,7R,7aR)-6-(2-amino-6-propoxy-9H-purin-9-yl)-7-fluoro-7-methyl-2-oxidotetrahydro-4H-furo[3,2-d][1,3,2]dioxaphosphinin-2-yl)amino)propanoate; or a pharmaceutically acceptable salt thereof.

Compounds of particular interest include: (S)-methyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; (R)-isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; and (S)-isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate; or a pharmaceutically acceptable salt thereof.

One compound of interest has the following structure:

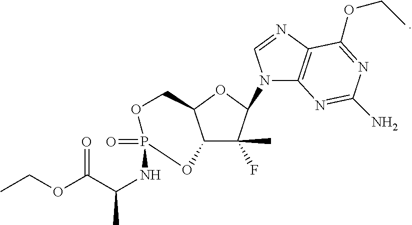 [SMILES: CCOC(=O)[C@H](C)N[P@@]1(=O)OC[C@H]2O[C@@H](n3cnc4c(OCC)nc(N)nc43)[C@](C)(F)[C@@H]2O1]

Another aspect of the present invention includes a pharmaceutical composition comprising a compound of Formula (I) which comprises any one of embodiments described above, and a pharmaceutically acceptable carrier or excipient. The pharmaceutical composition may further comprise at least one additional pharmaceutical agent described herein below. Examples of the additional pharmaceutical agent include, but are not limited to, interferons, ribavirin and ribavirin analogs, cyclophilin binder, HCV NS3 protease inhibitors, HCV NS5a inhibitors, P7 inhibitor, entry inhibitor, NS4b inhibitor, alpha-glucosidase inhibitors, host protease inhibitors, immune modulators, kinase inhibitors which induce cytokines or chemokines for severe dengue, symptomatic relief agents such as for plasma leakage etc., surface receptors such as CLEC5A and DC-SIGN, nucleoside and non-nucleoside NS5b inhibitors.

In yet another aspect of the present invention, a method is provided for treating a disease caused by a viral infection comprising the step of administering to a subject (in particular, a human) in need thereof, a therapeutically effective amount of a compound of Formula (I) including any of the embodiments described herein. In a particular useful embodiment, the viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus. In a more particular useful embodiment, the viral infection is caused by dengue virus. The compound may be administered as a pharmaceutical composition described herein

Another aspect of the present invention includes a compound of Formula (I) comprising any one of the embodiments described above, for use as a medicament (e.g., the use of a compound of Formula (I) comprising any one of the embodiments described above in the manufacture of a medicament for treating a disease caused by a viral infection). In a particular useful embodiment, the viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus. In a more particular useful embodiment, the viral infection is caused by dengue virus.

Another aspect of the present invention includes polymorphic forms of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate which has the below structure.

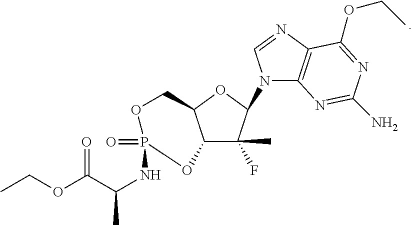 [SMILES: CCOC(=O)[C@H](C)N[P@@]1(=O)OC[C@H]2O[C@@H](n3cnc4c(OCC)nc(N)nc43)[C@](C)(F)[C@@H]2O1]

In one embodiment, the present invention provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 1 (referred to herein as “Form I”). The present invention also provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form I”) having a powder X-ray diffraction pattern comprising peaks at diffraction angles (2θ) of 7.6°, 10.3°, 11.1°, 11.8°, 12.3°, 15.2°, 16.5°, 18.1°, 19.9°, 20.7°, 21.5°, 22.2°, 23.6°, 25.3°, 25.7° and 29.5°.

In another embodiment, the present invention provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 4 (referred to herein as “Form II”). The present invention also provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form II”) having a powder X-ray diffraction pattern comprising peaks at diffraction angles (2θ) of 8.1°, 10.8°, 11.4°, 12.2°, 12.7°, 14.5°, 15.6°, 18.1°, 19.1°, 20.1°, 20.3°, 21.7°, 22.7°, 23.0°, 23.7°, 24.4°, 25.3°, 25.7° and 27.2°.

In yet another embodiment, the present invention provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 7 (referred to herein as “Form III”). The present invention also provides a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form III”) having a powder X-ray diffraction pattern comprising peaks at diffraction angles (2θ) of 7.7°, 12.3°, 15.5°, 16.6°, 17.4°, 20.0°, 22.1°, 22.9°, 24.6° and 35.6°.

Preferably, the crystalline forms described above (Form I, Form II and Form III) are substantially pure.

In yet another embodiment, the present invention provides a pharmaceutical composition comprising a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate in accordance with any one of the crystalline forms described above (Form I, Form II and Form III); and a pharmaceutically acceptable excipient, diluent or carrier. The pharmaceutical composition can further comprise at least one additional pharmaceutical agent. The additional pharmaceutical agent can be selected from the group consisting of interferons, ribavirin and ribavirin analogs, cyclophilin binder, HCV NS3 protease inhibitors, HCV NS5a inhibitors, P7 inhibitor, entry inhibitor, NS4b inhibitor, alpha-glucosidase inhibitors, host protease inhibitors, immune modulators, symptomatic relief agents, nucleoside and non-nucleoside NS5b inhibitors.

In another embodiment, the present invention provides a method of treating a disease caused by a viral infection comprising the step of administering to a mammal in need thereof a therapeutically effective amount of a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate in accordance with any one of the crystalline forms described above (Form I, Form II and Form III), or a pharmaceutical composition thereof. The viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

In yet another embodiment, the present invention provides the use of a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate in accordance with any one of the crystalline forms described above (Form I, Form II and Form III) in the treatment of dengue fever.

### DEFINITIONS

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, a subject is “in need of” a treatment if such subject would benefit biologically, medically or in quality of life from such treatment (preferably, a human).

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

The phrase “pharmaceutically acceptable” indicates that the substance or composition must be compatible chemically and/or toxicologically, with the other ingredients comprising a formulation, and/or the mammal being treated therewith.

The term “compounds of the present invention” (unless specifically identified otherwise) refer to compounds of Formulae (I)-(V) and salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates). For purposes of this invention, solvates and hydrates are generally considered compositions.

The term “a,” “an,” “the” and similar terms used in the context of the present invention (especially in the context of the claims) are to be construed to cover both the singular and plural unless otherwise indicated herein or clearly contradicted by the context.

As used herein, the term “substantially pure” with reference to a particular polymorphic form means that the polymorphic form includes less than 10%, preferably less than 5%, more preferably less than 3%, most preferably less than 1% by weight of any other physical forms of the compound.

The term “substantially the same” with reference to X-ray diffraction peak positions means that typical peak position and intensity variability are taken into account. For example, one skilled in the art will appreciate that the peak positions (2θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only.

### BRIEF DESCRIPTION OF THE DRAWINGS

FIG. 1 shows a powder X-ray diffraction pattern of the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form I”).

FIG. 2 shows a differential scanning calorimetry (DSC) thermogram of the “Form I” polymorph of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate.

FIG. 3 shows a TGA thermogram of the “Form I” polymorph of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate.

FIG. 4 shows a powder X-ray diffraction pattern of the crystalline fom of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form II”).

FIG. 5 shows a differential scanning calorimetry (DSC) thermogram of the “Form II” polymorph of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate.

FIG. 6 shows a TGA thermogram of the “Form II” polymorph of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate.

FIG. 7 shows a powder X-ray diffraction pattern of a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form III”).

FIG. 8 shows a differential scanning calorimetry (DSC) thermogram for the “Form III” polymorph of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate.

FIG. 9 shows the powder X-ray diffraction patterns overlay of slurry competition studies of crystalline forms of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form I, II and III) in 2-propanol at room temperature.

### DETAILED DESCRIPTION OF THE INVENTION

The present invention provides compounds and pharmaceutical compositions thereof that are useful in treating a disease caused by a viral infection, in particular viral infection caused by dengue virus.

Compounds of the present invention may be synthesized by synthetic routes that include processes analogous to those well-known to those of skill in the art, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Scheme 1 (below) describes a potential route for producing compounds of Formula (I). Compounds of Formula (I) can be made substantially optically pure by either using substantially optically pure starting material or by separation chromatography, recrystallization or other separation techniques well-known in the art. For a more detailed description, see the Example section below.

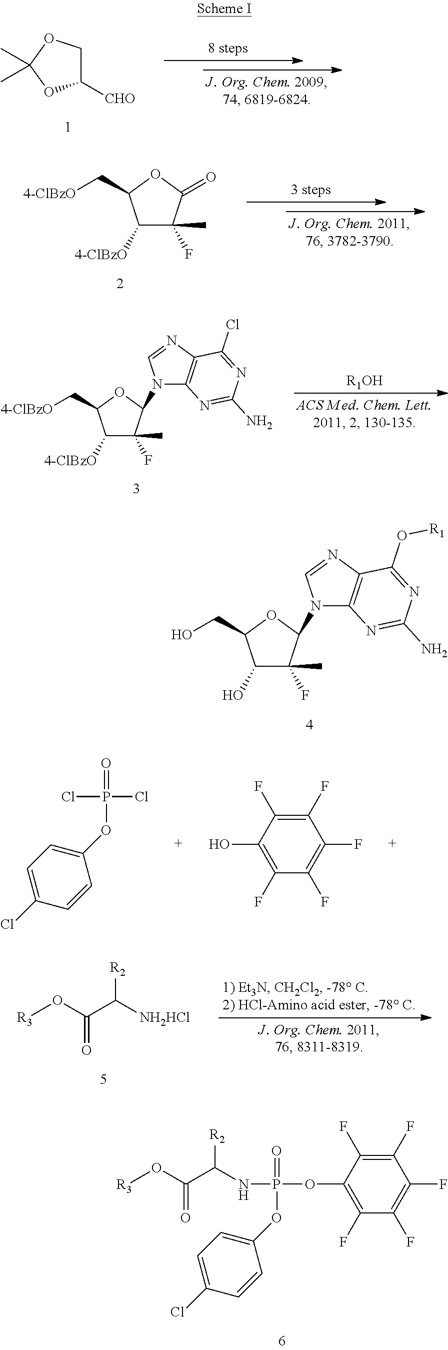

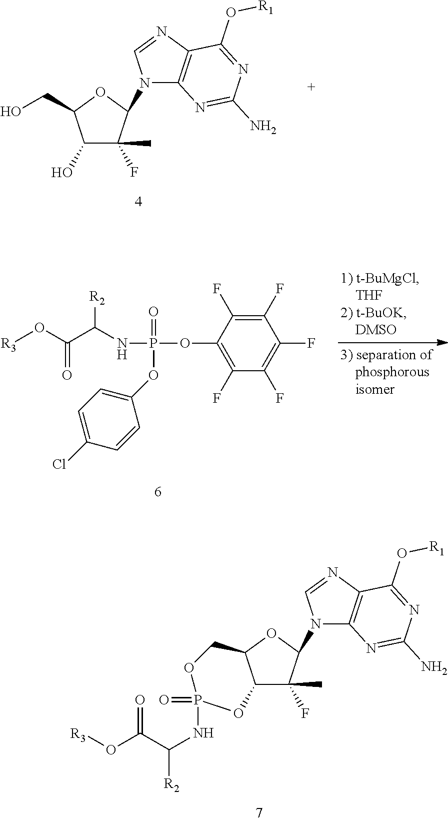

Compound 2 was synthesized from compound 1 according to the synthesis procedures described in Wang, P. et al. J. Org. Chem. 2009, 74, 6819-6824. Compound 2 was converted to compound 3 according to the synthesis procedures described in Reddy, P. G. et al. J. Org. Chem. 2011, 76, 3782-3790. Compound 4 was prepared by alkolysis of compound 3 according to the synthesis procedures described in Chang, W. et al. ACS Med. Chem. Lett. 2011, 2, 130-135. Compound 6 was prepared from compound 5 according to the synthesis procedures described in Ross, B. S. et al. J. Org. Chem. 2011, 76, 8311-8319. Compound 6 can be isomerized to be single Sp isomer by re-crystallization. Compound 6 was dissolved in a suitable solvent such as DMSO, acetonitrile, N-methylpyrrolidone, or DMF and treated with a suitable base such as potassium tert-butoxide or sodium tert-butoxide, and with Compound 4 to form Compound 7. Compound 7 was subject to preparative HPLC to afford various single stereoisomers of Compound 7.

The compounds and intermediates may be isolated and used as the compound per se. Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and, such as ²H, ³H, ¹¹C, ¹³C, ¹⁴C, ¹⁵N, ¹⁸F, ³¹P, ³²P, respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F, ¹¹C or labeled compound may be particularly desirable for PET or SPECT studies.

Further, substitution with heavier isotopes, particularly deuterium (i.e., ²H or D) may afford certain therapeutic advantages resulting from greater metabolic stability, for example increased in vivo half-life, reduced dosage requirements, reduced CYP450 inhibition (competitive or time dependent) or an improvement in therapeutic index. For example, substitution with deuterium may modulate undesirable side effects of the undeuterated compound, such as competitive CYP450 inhibition, time dependent CYP450 inactivation, etc. It is understood that deuterium in this context is regarded as a substituent in compounds of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of the present invention can generally be prepared by conventional techniques known to those skilled in the art or by carrying out the procedures disclosed in the schemes or in the examples and preparations described below using an appropriate isotopically-labeled reagent in place of the non-isotopically labeled reagent.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different isomeric forms. As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate.

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present invention are meant to include all such possible isomers, including racemic mixtures, optically pure forms and intermediate mixtures. Optically active (R)- and (S)-isomers may be prepared using chiral synthons or chiral reagents, or resolved using conventional techniques. All tautomeric forms are also intended to be included.

Mixtures of isomers obtainable according to the invention can be separated in a manner known to those skilled in the art into the individual isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Compounds of the invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In certain instances, it may be advantageous to administer the compound of the present invention in combination with at least one additional pharmaceutical (or therapeutic) agent. The compound of the present invention may be administered either simultaneously with, or before or after, one or more other therapeutic agent(s).

Alternatively, the compound of the present invention may be administered separately, by the same or different route of administration, or together in the same pharmaceutical composition as the other agent(s).

Suitable additional pharmaceutical agents include, but not limited to, interferons, ribavirin and ribavirin analogs, cyclophilin binder, HCV NS3 protease inhibitors, HCV NS5a inhibitors, P7 inhibitor, entry inhibitor, NS4b inhibitor, alpha-glucosidase inhibitors, host protease inhibitors, immune modulators, kinase inhibitors which induce cytokines or chemokines for severe dengue, symptomatic relief agents such as for plasma leakage etc., surface receptors such as CLEC5A and DC-SIGN, nucleoside and non-nucleoside NS5b inhibitors.

The compound of the present invention or pharmaceutical composition thereof for use in humans is typically administered orally at a therapeutic dose.

It will be appreciated that the dosage range of a compound of the invention to be employed for treating a viral infection depends upon factors known to the person skilled in the art, including host, nature and severity of the condition to be treated, the mode of administration and the particular substance to be employed.

The daily dosage of the compound of the invention will vary with the compound employed, the mode of administration, the treatment desired and the disease indicated, as well as other factors such as a subject's age, body weight, general health, condition, prior medical history and sex, and like factors known in the medical arts. For example, a compound of the invention is administered at a daily dosage in the range from about 0.5 mg/kg body weight to about 15 mg/kg body weight, e.g. in the range from about 1 mg/kg body weight to about 10 mg/kg body weight. Typically, satisfactory results can be obtained when the compound of the invention is administered at a daily dosage from about 0.001 g to about 10 g, e.g. not exceeding about 1 gram, e.g. from about 0.1 g to about 0.5 g for a 70 kg human, given up to 4 times daily.

Furthermore, several divided dosages, as well as staggered dosages, can be administered daily or sequentially, or the dose can be continuously infused, or can be a bolus injection. Further, the dosages of the compounds of the invention can be proportionally increased or decreased as indicated by the exigencies of the therapeutic or prophylactic situation.

In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

Another aspect of the invention is a product comprising a compound of the present invention and at least one other therapeutic agent (or pharmaceutical agent) as a combined preparation for simultaneous, separate or sequential use in therapy to treat a subject having a disease caused by viral infection.

In the combination therapies of the invention, the compound of the present invention and the other therapeutic agent may be manufactured and/or formulated by the same or different manufacturers. Moreover, the compound of the present invention and the other therapeutic (or pharmaceutical agent) may be brought together into a combination therapy: (i) prior to release of the combination product to physicians (e.g. in the case of a kit comprising the compound of the invention and the other therapeutic agent or fixed dose composition); (ii) by the physician themselves (or under the guidance of the physician) shortly before administration; (iii) in the patient themselves, e.g. during sequential administration of the compound of the invention and the other therapeutic agent.

It is especially advantageous to formulate the pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Daily dosages with respect to the other therapeutic agent used will vary depending upon, for example, the compound employed, the host, the mode of administration and the severity of the condition to be treated. Because of the diverse types of the other therapeutic agent that may be used, the amounts can vary greatly, and can be determined by routine experimentation, as described above.

The compound of the invention and at least one other therapeutic (or pharmaceutical) agent may be administered by any conventional route, in particular enterally, e.g. orally, for example in the form of solutions for drinking, tablets or capsules or parenterally, for example in the form of injectable solutions or suspensions.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and International Application Publication No. WO 95/13090, the disclosures of which are incorporated herein. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-α product. Pegylated interferon-α-2a is described e.g. in European Patent 593,868 (incorporated herein by reference in its entirety) and commercially available e.g. under the tradename PEGASYS® (Hoffmann-La Roche). Pegylated interferon-α-2b is described, e.g. in European Patent 975,369 (incorporated herein by reference in its entirety) and commercially available e.g. under the tradename PEG-INTRON A® (Schering Plough). Pegylated consensus interferon is described in WO 96/11953 (incorporated herein by reference in its entirety). The preferred pegylated α-interferons are pegylated interferon-α-2a and pegylated interferon-α-2b. Also preferred is pegylated consensus interferon.

Other preferred other therapeutic (or pharmaceutical) agents include fusion proteins of an interferon, for example fusion proteins of interferon-α-2a, interferon-α-2b; consensus interferon or purified interferon-α product, each of which is fused with another protein. Certain preferred fusion proteins comprise an interferon (e.g., interferon-α-2b) and an albumin as described in U.S. Pat. No. 6,973,322 and international publications WO02/60071, WO05/003296 and WO05/077042 (Human Genome Sciences). A preferred interferon conjugated to a human albumin is Albuferon (Human Genome Sciences).

Cyclosporins which bind strongly to cyclophilin but are not immunosuppressive include those cyclosporins recited in U.S. Pat. Nos. 5,767,069 and 5,981,479 and are incorporated herein by reference. [Melle]⁴-cyclosporin is a preferred non-immunosuppressive cyclosporin. Certain other cyclosporin derivatives are described in WO2006039668 (Scynexis) and WO2006038088 (Debiopharm SA) and are incorporated herein by reference. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway. [Melle]4-cyclosporin is a preferred non-immunosuppressive cyclophilin-binding cyclosporin for use according to the invention.

Ribavirin (1-β-D-ribofuranosyl-1-1,2,4-triazole-3-caroxamide) is a synthetic, non-interferon-inducing, broad spectrum antiviral nucleoside analog sold under the trade name Virazole (The Merck Index, 11^{th} edition, Editor: Budavar, S, Merck & Co., Inc., Rahway, N.J., p1304, 1989). U.S. Pat. No. 3,798,209 and RE29,835 disclose and claim ribavirin. Ribavirin is structurally similar to guanosine, and has in vitro activity against several DNA and RNA viruses including Flaviviridae (Gary L. Davis, Gastroenterology 118:S104-S114, 2000).

Other combinations include those of a compound of the invention with a non-immunosuppressive cyclophilin-binding cyclosporine, with mycophenolic acid, a salt or a prodrug thereof, and/or with a S1P receptor agonist, e.g. Fingolimod.

In another aspect, this invention provides a method comprising administering a compound of the invention and another anti-viral agent, preferably an anti-Flaviviridae, e.g. and anti-dengue or anti-Hepatitis C virus agent. Such anti-viral agents include, but are not limited to, immunomodulatory agents, such as α, β, and δ interferons, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the Flaviviridae (e.g. dengue virus, Hepatitis C virus) life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenolic acid and derivatives thereof, and including, but not limited to VX-497, VX-148, and/or VX-944); or combinations of any of the above.

Each component of a combination according to this invention may be administered separately, together, or in any combination thereof. As recognized by skilled practitioners, dosages of interferon are typically measured in IU (e.g., about 4 million IU to about 12 million IU). Each component may be administered in one or more dosage forms. Each dosage form may be administered to the subject in any order.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from a non-excluding commercial sources such as TCI Fine Chemicals (Japan), Shanghai Chemhere Co., Ltd. (Shanghai, China), Aurora Fine Chemicals LLC (San Diego, Calif.), FCH Group (Ukraine), Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Conier Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

The following abbreviations used herein below have the corresponding meanings:
h hour(s)
DCM dichloromethane
NMR nuclear magnetic resonance
TLC thin layer chromatography
MS mass spectrometry
LC-MS liquid chromatography-mass spectrometry
HPLC high performance liquid chromatography
DMSO dimethylsulfoxide
TEA triethylamine
DMF dimethylformamide
THF tetrahydrofuran
Na₂SO₄ sodium sulphate
HCl hydrochloric acid
EA ethyl acetate
ACN acetonitrile
t-Bu tert-butyl

### Example 1

### Preparation of (S)-ethyl 2-(((2R,4aR,6R,7R,7aR)-6-(2-amino-6-ethoxy-9H-purin-9-O-7-fluoro-7-methyl-2-oxidotetrahydro-4H-furo[3,2-d][1,3,2]dioxaphosphinin-2-yl)amino)propanoate

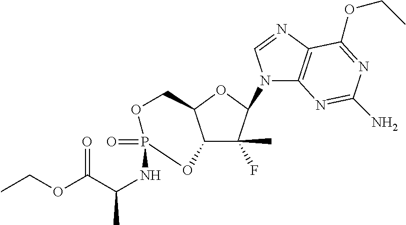 [SMILES: CCOC(=O)[C@H](C)N[P@@]1(=O)OC[C@H]2O[C@@H](n3cnc4c(OCC)nc(N)nc43)[C@](C)(F)[C@@H]2O1]

### Step 1

(2R,3R,4R,5R)-5-(2-amino-6-ethoxy-9H-purin-9-yl)-4-fluoro-2-(hydroxymethyl)-4-methyltetrahydrofuran-3-ol was prepared according to the procedures (e.g., scheme 6) as described in Reddy, P. G. et al. J. Org. Chem. 2011, 76, 3782-3790.

### Step 2: Synthesis of (2S)-ethyl 2-(((4-chlorophenoxy)(perfluorophenoxy)phosphoryl) amino)propanoate

To a stirred solution of 4-chlorophenyl phosphorodichloridate (25 g, 102 mmol) in DCM (500 mL) was added dropwise a solution of pentafluorophenol (18.7 g, 102 mmol) and triethyl amine (14.3 mL, 102 mmol) in DCM (200 mL) at −78° C. The reaction mixture was stirred at −78° C. for 3 h. A pre-stirred solution of L-alanine ethyl ester hydrochloride (18.7 g, 122 mmol) in DCM (500 mL) and triethylamine (42.9 mL, 306 mmol) was added dropwise to the reaction mixture at −78° C. The reaction mixture was stirred at −78° C. for 2 h. The reaction mixture was poured into ice-cold water and the organic layer was separated. The aqueous layer was extracted with DCM. The combined organic extracts were dried over Na₂SO₄, filtered, and concentrated. The residue was purified by silica gel column chromatography (the title compound was eluted at around 10-15% EA in n-hexane as white solids). The same scale reaction was repeated. The combined product was recrystallized using EA-n-pentane to afford 26.2 g of the title compound as white solids (Yield 27%) as a single Sp isomer.

¹H NMR (400 MHz, DMSO-d₆) δ 1.15 (t, J=7.2 Hz, 3H), 1.27 (d, J=7.2 Hz, 3H), 3.93-4.00 (m, 1H), 4.06 (q, J=7.2 Hz, 2H), 6.98 (dd, J=14, 10 Hz, 1H), 7.25 (d, J=9.0 Hz, 2H), 7.49 (d, J=9.0 Hz, 2H).

³¹P NMR (162 MHz, METHANOL-d₄) δ 0.39.

### Step 3: Synthesis of (2S)-ethyl 2-(((((2R,3R,4R,5R)-5-(2-amino-6-ethoxy-9H-purin-9-yl)-4-fluoro-3-hydroxy-4-methyltetrahydrofuran-2-yl)methoxy)(4-chlorophenoxy)phosphoryl)amino)propanoate

(2R,3R,4R,5R)-5-(2-amino-6-ethoxy-9H-purin-9-yl)-4-fluoro-2-(hydroxymethyl)-4-methyltetrahydrofuran-3-ol (2 g, 6.11 mmol) was dissolved in THF (30 mL), cooled to −10° C. t-BuMgCl in THF (11.61 mL, 1 M, 11.61 mmol) was added at −10° C. under argon atmosphere, a clear solution formed. The reaction mixture was stirred at −10° C. for 30 minutes, then cooled down to −35° C. by adding dry ice pellets to the actone bath. (2S)-ethyl 2-(((4-chlorophenoxy)(perfluorophenoxy)phosphoryl)amino)propanoate (5.79 g, 2.22 mmol) in THF (15 mL) was added to the reaction mixture at −35° C. in 20 minutes. The reaction mixture was allowed to slowly warm up to −10° C. in 2 h, then stirred at 0° C. for 1 h. The reaction was quenched with saturated NH₄Cl solution (100 mL) at 0° C. and extracted with EA (3×60 mL). The combined EA layer was washed with brine, dried over anhydrous Na₂SO₄, filtered, concentrated, which was then purified by flash column (MeOH/DCM 0% to 15% gradient) again to afford 4.11 g of the title compound as white solid (Yield 83%).

¹H NMR (400 MHz, DMSO-d₆) δ 1.15-1.05 (m, 6H), 1.20 (d, J=7.20 Hz, 3H), 1.36 (t, J=7.20 Hz, 3H), 4.15-3.75 (m, 4H), 4.55-4.20 (m, 4H), 6.15-6.05 (m, 2H), 6.57 (s, 2H), 7.23 (d, J=8.0 Hz, 2H), 7.42 (d, J=8.0 Hz, 2H), 7.96 (s, 1H). ³¹P NMR (162 MHz, DMSO-d₆) δ 3.93.

### Step 4: (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

A suspension of KO^{t}Bu (0.498 g, 4.43 mmol) in DMSO (25 mL) was warmed up and sonicated, then at 25° C. was added (2S)-ethyl 2-((((2R,3R,4R,5R)-5-(2-amino-6-ethoxy-9H-purin-9-yl)-4-fluoro-3-hydroxy-4-methyltetrahydrofuran-2-yl)methoxy)(4-chlorophenoxy)phosphoryl)amino)propanoate (3 g, 3.70 mmol). The reaction was stirred at room temperature for 10 minutes and quenched with 1N HCl solution (˜8 mL) at 25° C. to adjust the pH to 4-6, and stirred at room temperature for 10 minutes. ACN (15 mL) was added to dilute the solution for PREP-HPLC, inorganic salt was filtered. The crude product was directly purified by PREP-HPLC (60% to 95% MeOH in H₂O in 8 minutes using SUNFIRE column, flow rate: 20 mL/minutes). Desired fast eluting isomer (Rp product) was combined and concentrated to dry to afford 1.012 g of the title compound (Yield 55%).

¹H NMR (400 MHz, METHANOL-d₄) δ 1.32 (t, J=6.8 Hz, 3H), 1.34 (d, J=22.0 Hz, 3H), 1.45 (t, J=6.8 Hz, 3H), 1.50 (d, J=7.2 Hz, 3H), 3.98 (m, 1H), 4.26 (q, J=7.2 Hz, 2H), 4.39 (m, 1H), 4.56 (q, J=7.2 Hz, 2H), 4.63 (m, 2H), 5.70 (br, 1H), 6.24 (d, J=20.0 Hz, 1H), 7.96 (s, 1H).

³¹P NMR (162 MHz, METHANOL-d₄) δ 5.26.

MS (m+1)=489.

### Example 2

### Preparation of (S)-isopropyl 2-(((2S,4aR,6R,7R,7aR)-6-(2-amino-6-ethoxy-9H-purin-9-yl-7-fluoro-7-methyl-2-oxidotetrahydro-4H-furo[3,2-d][1,3,2]dioxaphosphinin-2-yl)amino)propanoate

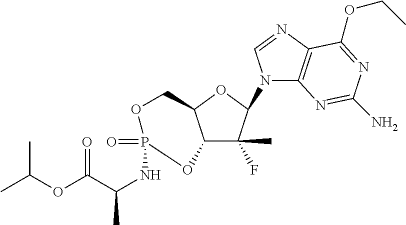 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F]

### Step 1: Synthesis of (2S)-isopropyl 2-(((4-chlorophenoxy)(perfluorophenoxy)phosphoryl)amino)propanoate

To a stirred solution of 4-chlorophenyl phosphorodichloridate (1.989 mL, 12.22 mmol) in DCM (20 mL) was added a solution of pentafluorophenol (2.250 g, 12.22 mmol) and triethyl amine (1.546 mL, 12.22 mmol) in DCM (20 mL) at −78° C. over a period of 20 minutes. The reaction mixture was maintained at −78° C. for 1.5 h. L-alanine iso-propyl ester hydrochloride (2.049 g, 12.22 mmol) in DCM (20 mL) was added at −78° C., followed by triethyl amine (3.09 mL, 24.45 mmol). The reaction mixture was maintained at −78° C. for 1 h, then allowed to warm to 0° C., and maintained at 0° C. for another 1 h. The reaction mixture was filtered and the filtrate was concentrated and purified by flash column (the title compound was eluted at around 18-22% ethyl acetate in Cyclohexane) which afforded 2.6 g of the title compound as white solids as 1:1 mixture of Sp and Rp isomers (Yield 39%).

¹H NMR (400 MHz, METHANOL-d₄): δ 1.24 (dd, J=6.27, 2.51 Hz, 6H), 1.34-1.45 (m, 3H), 4.04 (dd, J=10.04, 7.28 Hz, 1H), 4.93-5.06 (m, 1H), 7.22-7.36 (m, 2H), 7.39-7.48 (m, 2H).

³¹P NMR (162 MHz, METHANOL-d₄): δ 0.27, 0.38.

### Step 2: Synthesis of (2S)-isopropyl 2-(((((2S,3R,4R,5R)-5-(2-amino-6-ethoxy-9H-purin-9-yl)-4-fluoro-3-hydroxy-4-methyltetrahydrofuran-2-yl)methoxy)(4-chlorophenoxy)phosphoryl)amino)propanoate

(2R,3R,4R,5R)-5-(2-amino-6-ethoxy-9H-purin-9-yl)-4-fluoro-2-(hydroxymethyl)-4-methyltetrahydrofuran-3-ol (0.73 g, 2.230 mmol) was dissolved in THF (30 mL), cooled to −20° C., tert-butylmagnesium chloride in THF (4.46 mL, 1 M, 4.46 mmol) was added at −20° C., and the reaction mixture was maintained at −20° C. for 20 minutes. (2S)-isopropyl 2-(((4-chlorophenoxy)(perfluorophenoxy)phosphoryl)amino)propanoate (2.176 g, 4.46 mmol) was added, the reaction mixture was allowed to warm to 0° C. for 4 h. LC-MS and TLC showed the completion of the reaction. The reaction mixture was poured into cold water and extracted with EA. The combined organic extracts were dried over anhydrous Na₂SO₄, filtered, and concentrated. The residue was purified by flash column (the title compound eluted at 9-11% methanol in DCM) to afford 1.2 g of the title compound as white solid (Yield 72%). Proton NMR showed the title compound is a mixture of two phosphorous diasteroisomers and the ratio of the two diasteroisomers is 1:1.

¹H NMR (400 MHz, METHANOL-d₄): δ 1.09-1.36 (m, 12H), 1.39-1.52 (m, 3H), 3.82-3.96 (m, 1H), 4.16-4.29 (m, 1H), 4.48-4.65 (m, 4H), 4.89-5.00 (m, 1H), 6.18 (dd, J=18.70, 9.66 Hz, 1H), 7.18-7.25 (m, 2H), 7.27-7.37 (m, 2H), 7.95 (d, J=6.02 Hz, 1H).

³¹P NMR (162 MHz, METHANOL-d₄) δ 4.00.

### Step 3: Synthesis of (S)-isopropyl 2-(((2S,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

The compound obtained from step 2 (1.5 g, 2.377 mmol) was dissolved in DMSO (30 mL), potassium tert-butoxide (267 mg, 2.377 mmol) was added at 22° C. The reaction mixture was stored at room temperature for 30 minutes. LC-MS showed the completion of the reaction. 1N HCl (3 mL) was added to adjust the pH to 6-7. The mixture was purified by PREP-HPLC (40% to 95% MeOH in H₂O, ATIANTIS COLUMN, flow rate: 20 mL/minute, the title compound eluted at 18 minutes) to afford 440 mg of the title compound as white solid (Yield 35%) as a Sp isomer as well as 210 mg (Yield 17%) as a Rp isomer.

Sp isomer (title compound)

¹H NMR (400 MHz, METHANOL-d₄) δ 1.21-1.52 (m, 15H), 3.83-3.96 (m, 1H), 4.24-4.36 (m, 1H), 4.49-4.70 (m, 4H), 5.04 (dt, J=12.49, 6.18 Hz, 1H), 5.61-5.84 (m, 1H), 6.23 (d, J=20.6 Hz, 1H), 7.95 (s, 1H).

³¹P NMR (162 MHz, METHANOL-d₄) δ 7.60 ppm.

MS (m+1)=503.

Rp isomer

¹H NMR (400 MHz, METHANOL-d₄) δ 1.18-1.56 (m, 15H), 3.93 (dd, J=9.66, 7.15 Hz, 1H), 4.30-4.46 (m, 1H), 4.49-4.69 (m, 4H), 5.09 (dt, J=12.55, 6.27 Hz, 1H), 5.73 (br. s., 1H), 6.25 (d, J=19.83 Hz, 1H), 7.96 (s, 1H).

³¹P NMR (162 MHz, METHANOL-d₄) δ 5.32.

MS (m+1)=503.

### Examples 3-15

The compounds in Table 1 below were prepared using the general procedures as well as the procedures from the examples described above with the appropriate starting materials.

TABLE 1

| Structure/Example No./Name | Analytical Data |
|---|---|
| 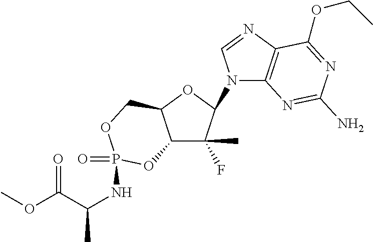 [SMILES: CCOc1nc(N)nc2c1ncn2[C@@H]1O[C@@H]2CO[P@](=O)(N[C@@H](C)C(=O)OC)O[C@H]2[C@@]1(C)F] Example 3 (S)-methyl 2-(((2R,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2- yl)amino)propanoate | ¹H NMR (400 MHz, METHANOL-d4) δ ppm 1.34 (d, J = 22.3 Hz, 3H), 1.46 (t, J = 7.1 Hz, 3H), 1.48-1.53 (m, 3H), 3.80 (s, 3H), 4.01 (dd, J = 9.91, 7.15 Hz, 1H), 4.40 (br. s., 1H), 4.50-4.70 (m, 4H), 5.55-5.84 (m, 1H), 6.25 (d, J = 19.83 Hz, 1H), 7.96 (s, 1H). ³¹P NMR (162 MHz, METHANOL-d₄) δ ppm 5.19. MS (m + 1) = 475. |
| 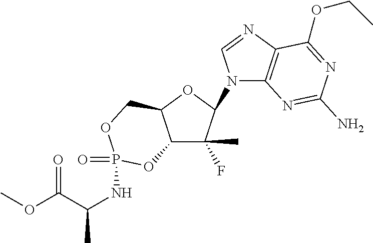 [SMILES: CCOc1nc(N)nc2c1ncn2[C@@H]1O[C@@H]2CO[P@@](=O)(N[C@@H](C)C(=O)OC)O[C@H]2[C@@]1(C)F] Example 4 (S)-methyl 2-(((2S,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2- yl)amino)propanoate | ¹H NMR (400 MHz, METHANOL-d4) δ ppm 1.27-1.55 (m, 9H), 3.76 (s, 3H), 3.97 (dq, J = 9.69, 7.14 Hz, 1H), 4.22-4.38 (m, 1H), 4.48-4.70 (m, 4H), 5.57- 5.89 (m, 1H), 6.23 (d, J = 20.0 Hz, 1H), 7.96 (s, 1 H). ³¹P NMR (162 MHz, METHANOL-d₄) δ ppm 7.54. MS (m + 1) = 475. |
| 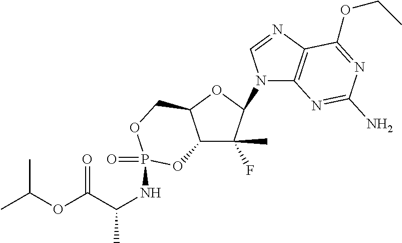 [SMILES: CCOc1nc(N)nc2c1ncn2[C@@H]1O[C@@H]2CO[P@](=O)(N[C@H](C)C(=O)OC(C)C)O[C@H]2[C@@]1(C)F] Example 5 (R)-isopropyl 2-(((2R,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2- yl)amino)propanoate | ¹H NMR (400 MHz, METHANOL-d4) δ ppm 1.27 (t, J = 6.0 Hz, 3H), 1.37 (d, J = 20.0 Hz, 3H), 1.44-1.50 (m, 6H), 3.92 (dq, J = 9.66, 7.15 Hz, 1H), 4.37 (tdd, J = 10.04, 10.04, 5.52, 1.76 Hz, 1H), 4.56 (q, J = 7.11 Hz, 2H), 4.59-4.77 (m, 2H), 5.03 (dt, J = 12.55, 6.27 Hz, 1H), 5.43-5.66 (m, 1H), 6.25 (d, J = 20.00 Hz, 1H), 7.95 (s, 1H). ³¹P NMR (162 MHz, METHANOL-d₄) δ ppm 4.51. MS (m + 1) = 503. |
| 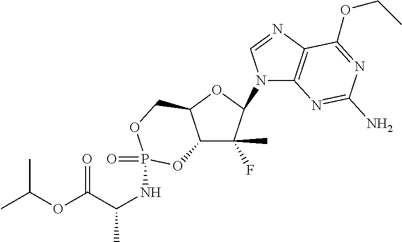 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] Example 6 (R)-isopropyl 2-(((2S,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2- yl)amino)propanoate | ¹H NMR(400 MHz, METHANOL-d4) δ ppm 1.29 (d, J = 6.15 Hz, 3H), 1.30 (d, J = 6.15 Hz, 3H), 1.37 (d, J = 24.0 Hz, 3H), 1.45 (t, J = 8.0 Hz, 3H), 1.40-1.42 (m, 3H), 3.90 (dd, J = 9.41, 7.15 Hz, 1 H), 4.25-4.36 (m, 1H), 4.55 (q, J = 7.03 Hz, 2H), 4.59-4.71 (m, 2H), 5.03 (dt, J = 12.55, 6.27 Hz, 1H), 5.59- 5.85 (m, 1H), 6.22 (d, J = 24.00 Hz, 1H), 7.95 (s, 1H). ³¹P NMR (162 MHz, METHANOL-d₄) δ ppm 7.66. MS (m + 1) = 503. |
| 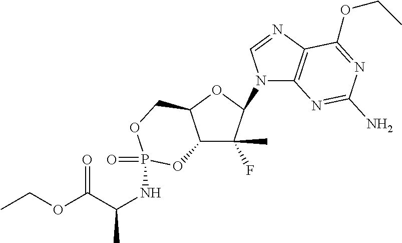 [SMILES: CCOC(=O)[C@H](C)N[P@]1(=O)OC[C@H]2O[C@@H](n3cnc4c(OCC)nc(N)nc43)[C@](C)(F)[C@@H]2O1] Example 7 (S)-ethyl 2-(((2S,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2- yl)amino)propanoate | ¹H NMR (400 MHz, METHANOL-d₄) δ ppm 1.31 (t, J = 8.0 Hz, 3H), 1.40 (d, J = 20.0 Hz, 3H), 1.42-1.44 (m, 3H), 1.45 (t, J = 8.0 Hz, 3H), 3.87-4.00 (m, 2H), 4.22 (q, J = 7.19 Hz, 2H), 4.26-4.35 (m, 1H), 4.49-4.60 (m, 3H), 4.64 (d, J = 5.77 Hz, 1H), 5.71 (br. s., 1H), 6.23 (d, J = 24.00 Hz, 1H), 7.95 (s, 1H). ³¹PNMR (162 MHz, METHANOL-d₄) δ ppm 7.59. MS (m + 1) = 489. |
| 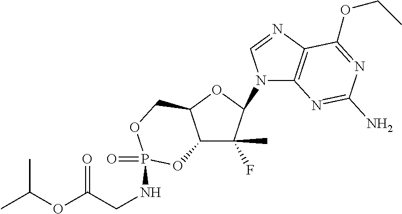 [SMILES: CCOc1nc(N)nc2c1ncn2[C@@H]1O[C@@H]2CO[P@](=O)(NCC(=O)OC(C)C)O[C@H]2[C@@]1(C)F] Example 8 isopropyl 2-(((2R,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2-yl)amino)acetate | ¹H NMR(400 MHz, METHANOL-d₄) δ ppm 1.29 (t, J = 8.0 Hz, 6H), 1.36 (d, J = 24.0 Hz, 3H), 1.46 (t, J = 8.0 Hz, 3H), 3.77 (d, J = 13.05 Hz, 2H), 4.39 (m, 1H), 4.56 (q, J = 7.03 Hz, 2H), 4.62-4.75 (m, 3H), 5.07 (dt, J = 12.55, 6.27 Hz, 1H), 6.27 (d, J = 20.0 Hz, 1H), 7.96 (s, 1H). ³¹P NMR (162 MHz, METHANOL-d₄) δ ppm 6.08. MS (m + 1) = 489. |
| 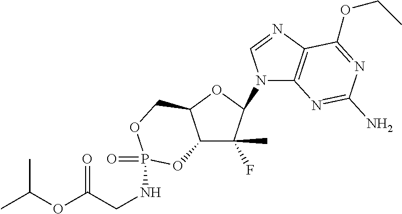 [SMILES: CCOc1nc(N)nc2c1ncn2[C@@H]1O[C@@H]2CO[P@@](=O)(NCC(=O)OC(C)C)O[C@H]2[C@@]1(C)F] Example 9 isopropyl 2-(((2S,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2-yl)amino)acetate | ¹H NMR (400 MHz, METHANOL-d₄) δ ppm 1.29 (d, J = 8.0 Hz, 6H), 1.39 (d, J = 20.0 Hz, 3H), 1.45 (t, J = 8.0 Hz, 3H), 3.72 (d, J = 12.0 Hz, 2H), 4.28-4.34 (m, 1H), 4.55 (q, J = 8.0 Hz, 2H), 4.65-4.67 (m, 3H), 5.06 (dt, J = 12.55, 6.27 Hz, 1H), 6.23 (d, J = 20.0 Hz, 1H), 7.95 (s, 1H). ³¹P NMR (162 MHz, METHANOL-d₄) δ ppm 8.84. MS (m + 1) = 489. |
| 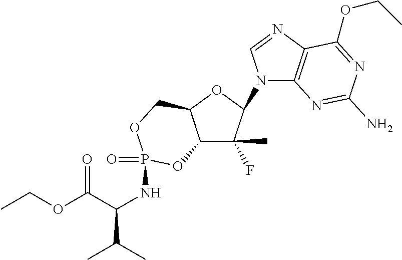 [SMILES: CCOC(=O)[C@@H](N[P@@]1(=O)OC[C@H]2O[C@@H](n3cnc4c(OCC)nc(N)nc43)[C@](C)(F)[C@@H]2O1)C(C)C] Example 10 (S)-ethyl 2-(((2R,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2-yl)amino)-3- methylbutanoate | ¹H NMR (400 MHz, METHANOL-d₄) δ ppm 1.03 (dd, J = 6.8, 0.7 Hz, 3H), 1.30- 1.35 (m, 6H), 1.46 (t, J = 7.1 Hz, 3H), 2.15 (dq, J = 13.4, 6.7 Hz, 1H), 3.65 (dd, J = 10.3, 6.1 Hz, 1H), 4.27 (qt, J = 7.0, 3.6 Hz, 2H), 4.41 (m, 1H), 4.56 (q, J = 7.1 Hz, 2H), 4.63 (m, 2H), 5.63 (br, 1H), 6.26 (d, J = 19.6 Hz, 1H), 7.96 (s, 1H). ³¹P NMR (162 MHz, METHANOL-d₄) δ ppm 5.77. MS (m + 1) = 517. |
| 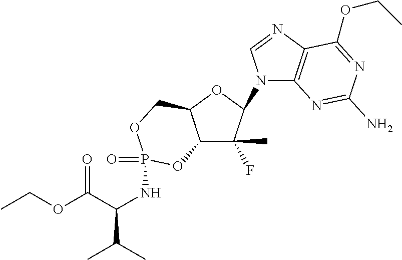 [SMILES: CCOC(=O)[C@@H](N[P@]1(=O)OC[C@H]2O[C@@H](n3cnc4c(OCC)nc(N)nc43)[C@](C)(F)[C@@H]2O1)C(C)C] Example 11 (S)-ethyl 2-(((2S,4aR,6R,7R,7aR)-6-(2- amino-6-ethoxy-9H-purin-9-yl)-7-fluoro-7- methyl-2-oxidotetrahydro-4H-furo[3,2- d][1,3,2]dioxaphosphinin-2-yl)amino)-3- methylbutanoate | ¹H NMR (400 MHz, METHANOL-d₄) δ ppm 0.99 (dd, J = 9.0, 6.8 Hz, 6 H), 1.31 (t, J = 7.1 Hz, 3H), 1,38 (d, J = 22.5 Hz, 3H), 1.45 (t, J = 7.1 Hz, 3H), 2.08 (m, 1H), 3.63 (dd, J = 10.4, 6.3 Hz, 1H), 4.23 (qd, J = 7.1, 1.4 Hz, 2H), 4.27-4.34 (m, 1 H), 4.55 (q, J = 7.1 Hz, 2H), 4.55-4.59 (m, 1H), 4.63-4.68 (m, 1H), 5.72 (br. s., 1H), 6.23 (d, J = 20.00 Hz, 1H), 7.95 (s, 1H). ³¹P NMR (162 MHz, METHANOL-d₄) δ ppm 8.36. MS (m + 1) = 517. |
| 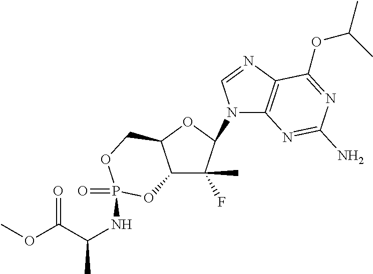 [SMILES: COC(=O)[C@H](C)N[P@@]1(=O)OC[C@H]2O[C@@H](n3cnc4c(OC(C)C)nc(N)nc43)[C@](C)(F)[C@@H]2O1] Example 12 (S)-methyl 2-(((2R,4aR,6R,7R,7aR)-6-(2- amino-6-isopropoxy-9H-purin-9-yl)-7-fluoro- 7-methyl-2-oxidotetrahydro-4H-furo[3,2- d][1,3,2]dioxaphosphinin-2- yl)amino)propanoate | ¹H NMR (400 MHz, METHANOL-d₄): δ ppm 1.35 (d, J = 20.0 Hz, 3H), 1.38-1.45 (m, 6H), 1.50 (d, J = 7.28 Hz, 3H), 3.80 (s, 3H), 3.88-4.10 (m, 1H), 4.29-4.45 (m, 1H), 4.55-4.75 (m, 2H), 4.80-4.95 (m, 1H), 5.45-5.85 (br, 1H), 6.27 (d, J = 20.0 Hz, 1H), 7.95 (s, 1H). ³¹P NMR (162 MHz, METHANOL-d₄): δ ppm 5.20. MS (m + 1) = 489.. |
| 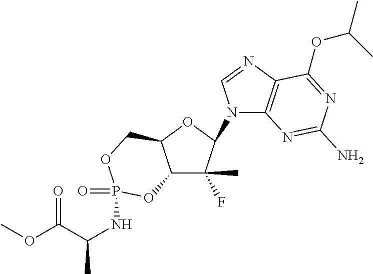 [SMILES: COC(=O)[C@H](C)N[P@]1(=O)OC[C@H]2O[C@@H](n3cnc4c(OC(C)C)nc(N)nc43)[C@](C)(F)[C@@H]2O1] Example 13 (S)-methyl 2-(((2S,4aR,6R,7R,7aR)-6-(2- amino-6-isopropoxy-9H-purin-9-yl)-7-fluoro- 7-methyl-2-oxidotetrahydro-4H-furo[3,2- d][1,3,2]dioxaphosphinin-2- yl)amino)propanoate | ¹H NMR (400 MHz, METHANOL-d₄): δ ppm 1.30- 1.45 (m, 12H), 3.75 (s, 3H), 3.90-4.10 (m, 1H), 4.20-4.40 (m, 1H), 4.55-4.75 (m, 2H), 5.45-5.65 (m, 1H), 5.45-5.85 (br, 1H), 6.20 (d, J = 20.0 Hz, 1H), 7.92 (s, 1H). . ³¹P NMR (162 MHz, METHANOL-d₄): δ 7.52 ppm. MS (m + 1) = 489. |
| 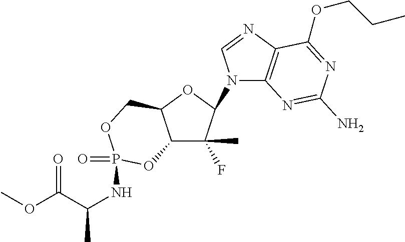 [SMILES: CCCOc1nc(N)nc2c1ncn2[C@@H]1O[C@@H]2CO[P@](=O)(N[C@@H](C)C(=O)OC)O[C@H]2[C@@]1(C)F] Example 14 (S)-methyl 2-(((2R,4aR,6R,7R,7aR)-6-(2- amino-6-propoxy-9H-purin-9-yl)-7-fluoro-7- methyl-2-oxidotetrahydro-4H-furo[3,2- d][1,3,2]dioxaphosphinin-2- yl)amino)propanoate | ¹H NMR(400 MHz, METHANOL-d₄): δ ppm 1.08 (t, J = 7.40 Hz, 3H), 1.34 (d, J = 22.0 Hz, 3H), 1.50 (d, J = 7.28 Hz, 3H), 1.80-1.95 (m, 2H), 3.80 (s, 3H), 3.95- 4.10 (m, 1H), 4.35-4.50 (m, 3H), 4.60-4.70 (m, 2H), 5.60- 5.90 (br, 1H), 6.25 (d, J = 20.0 Hz, 1H), 7.96 (s, 1H). ³¹P NMR (162 MHz, METHANOL-d₄): δ ppm 5.20. MS (m + 1) = 489. |
| 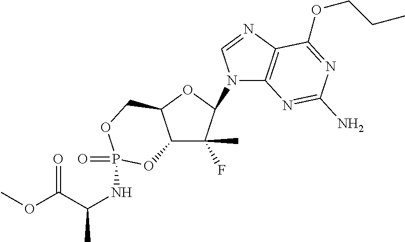 [SMILES: CCCOc1nc(N)nc2c1ncn2[C@@H]1O[C@@H]2CO[P@@](=O)(N[C@@H](C)C(=O)OC)O[C@H]2[C@@]1(C)F] Example 15 (S)-methyl 2-(((2S,4aR,6R,7R,7aR)-6-(2- amino-6-propoxy-9H-purin-9-yl)-7-fluoro-7- methyl-2-oxidotetrahydro-4H-furo[3,2- d][1,3,2]dioxaphosphinin-2- yl)amino)propanoate | ¹H NMR (400 MHz, METHANOL-d₄): δ ppm 1.07 (t, J = 8.00 Hz, 3H), 1.30-1.50 (m, 6H), 1.82-1.95 (m, 2H), 3.77 (s, 3H), 3.90-4.10 (m, 1H), 4.20-4.40 (m, 1H), 4.45 (t, J = 8.00 Hz, 3H), 4.50-4.75 (m, 2H), 5.60-5.85 (br, 1H), 6.23 (d, J = 20.0 Hz, 1H), 7.96 (s, 1H).. ³¹P NMR (162 MHz, METHANOL-d₄): δ ppm 7.53. MS (m + 1) = 489. |

Table 2 is a list of representative compounds of the invention (prepared as described above) with particular interest.

TABLE 2

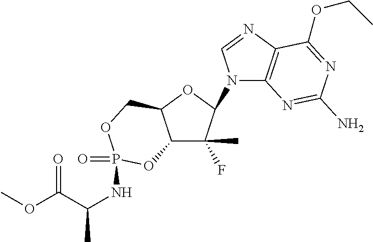 [SMILES: CCOc1nc(N)nc2c1ncn2[C@@H]1O[C@@H]2CO[P@](=O)(N[C@@H](C)C(=O)OC)O[C@H]2[C@@]1(C)F]

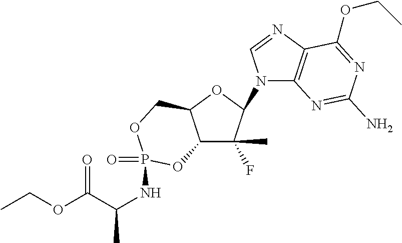 [SMILES: CCOC(=O)[C@H](C)N[P@@]1(=O)OC[C@H]2O[C@@H](n3cnc4c(OCC)nc(N)nc43)[C@](C)(F)[C@@H]2O1]

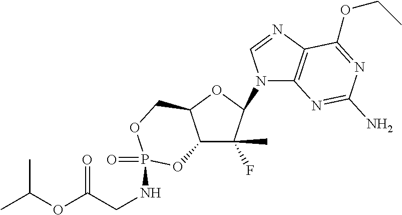 [SMILES: CCOc1nc(N)nc2c1ncn2[C@@H]1O[C@@H]2CO[P@](=O)(NCC(=O)OC(C)C)O[C@H]2[C@@]1(C)F]

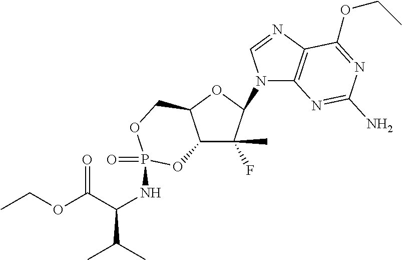 [SMILES: CCOC(=O)[C@@H](N[P@@]1(=O)OC[C@H]2O[C@@H](n3cnc4c(OCC)nc(N)nc43)[C@](C)(F)[C@@H]2O1)C(C)C]

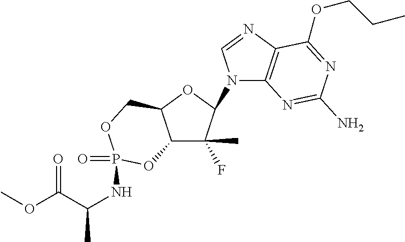 [SMILES: CCCOc1nc(N)nc2c1ncn2[C@@H]1O[C@@H]2CO[P@](=O)(N[C@@H](C)C(=O)OC)O[C@H]2[C@@]1(C)F]

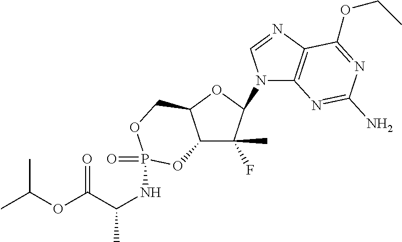 [SMILES: CCOc1nc(N)nc2c1ncn2[C@@H]1O[C@@H]2CO[P@@](=O)(N[C@H](C)C(=O)OC(C)C)O[C@H]2[C@@]1(C)F]

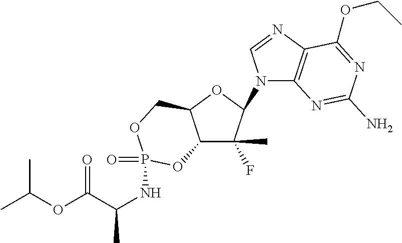 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F]

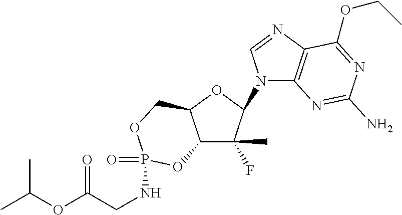 [SMILES: CCOc1nc(N)nc2c1ncn2[C@@H]1O[C@@H]2CO[P@@](=O)(NCC(=O)OC(C)C)O[C@H]2[C@@]1(C)F]

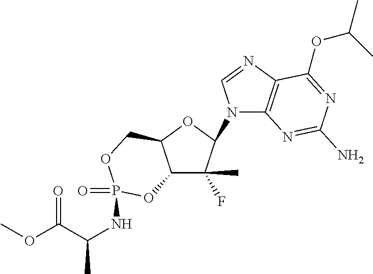 [SMILES: COC(=O)[C@H](C)N[P@@]1(=O)OC[C@H]2O[C@@H](n3cnc4c(OC(C)C)nc(N)nc43)[C@](C)(F)[C@@H]2O1]

Table 3 is a list of compounds for comparative purpose. The preparation of these compounds is provided below.

TABLE 3

| | |
|---|---|
| 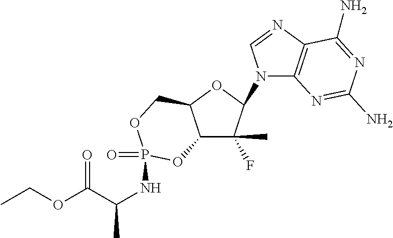 [SMILES: CCOC(=O)[C@H](C)N[P@@]1(=O)OC[C@H]2O[C@@H](n3cnc4c(N)nc(N)nc43)[C@](C)(F)[C@@H]2O1] | Comparative Example 1 |
| 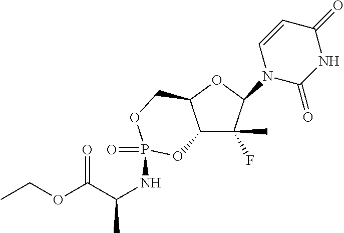 [SMILES: CCOC(=O)[C@H](C)N[P@@]1(=O)OC[C@H]2O[C@@H](n3ccc(=O)[nH]c3=O)[C@](C)(F)[C@@H]2O1] | Comparative Example 2 |
| 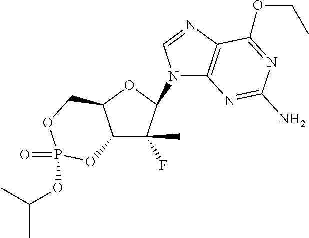 [SMILES: CCOc1nc(N)nc2c1ncn2[C@@H]1O[C@@H]2CO[P@@](=O)(OC(C)C)O[C@H]2[C@@]1(C)F] | Comparative Example 3 |
| 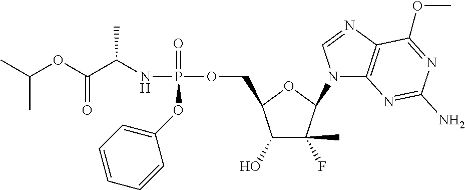 [SMILES: COc1nc(N)nc2c1ncn2[C@@H]1O[C@H](CO[P@@](=O)(N[C@@H](C)C(=O)OC(C)C)Oc2ccccc2)[C@@H](O)[C@@]1(C)F] | Comparative Example 4 |

### Comparative Example 1

### Preparation of (S)-Ethyl 2-(((2R,4aR,6R,7R,7aR)-6-(2,6-diamino-9H-purin-9-yl)-7-fluoro-7-methyl-2-oxidotetrahydro-4H-furo[3,2-d][1,3,2]dioxaphosphinin-2-yl)amino)propanoate

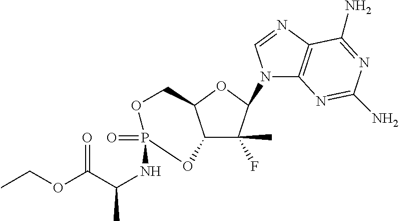 [SMILES: CCOC(=O)[C@H](C)N[P@@]1(=O)OC[C@H]2O[C@@H](n3cnc4c(N)nc(N)nc43)[C@](C)(F)[C@@H]2O1]

### Step 1: Synthesis of (2R,3R,4R,5R)-5-(2,6-diamino-9H-purin-9-yl)-4-fluoro-2-(hydroxymethyl)-4-methyltetrahydrofuran-3-ol

A mixture of (2R,3R,4R,5R)-5-(2-amino-6-chloro-9H-purin-9-yl)-2-(((4-chlorobenzoyl(oxy)methyl)-4-fluoro-4-methyltetrahydrofuran-3-yl 4-chlorobenzoate (prepared according to the procedures (e.g., scheme 6) as described in Reddy, P. G. et al. J. Org. Chem. 2011, 76, 3782-3790.) (2 g, 3.36 mmol) in 28% aqueous ammonia (30 mL) and 1,4-dioxane (10 mL) in was stirred at 110° C. in a sealed vessel for 13 h. After the mixture was concentrated, the residue was purified by silica gel column chromatography (DCM-MeOH) followed by crystallization from MeOH-EA to afford 666 mg of the title compound as white solid (Yield 66%).

¹H NMR (400 MHz, DMSO-d₆) δ 1.07 (d, J=24 Hz, 3H), 3.69 (ddd, J=12.4, 5.4, 3.5 Hz, 1H), 3.82-3.91 (m, 2H), 4.20 (dt, J=25.7, 8.2 Hz, 1H), 5.24 (t, J=5.1 Hz, 1H), 5.62 (d, J=7.2 Hz, 1H), 6.00 (d, J=18.3 Hz, 1H), 6.7 (s, 2H), 7.23 (s, 2H), 7.99 (s, 1H).

MS (m+1)=300.

### Step 2: Synthesis of (S)-ethyl 2-(((S)-(4-chlorophenoxy)(((2R,3R,4R,5R)-5-(2,6-diamino-9H-purin-9-yl)-4-fluoro-3-hydroxy-4-methyltetrahydrofuran-2-yl)methoxy)phosphoryl)amino)propanoate

(2R,3R,4R,5R)-5-[2,6-d]amino-9H-purin-9-yl)-4-fluoro-2-(hydroxymethyl)-4-methyltetrahydrofuran-3-ol (300 mg, 1.0 mmol) was dissolved in THF (4 mL), cooled to 0° C. t-BuMgCl in THF (1.6 mL, 1.6 mmol) was added at 0° C. under argon atmosphere. The reaction mixture was stirred vigorously at 0° C. for 30 minutes, (2S)-ethyl 2-(((4-chlorophenoxy)(perfluorophenoxy)phosphoryl) amino)propanoate in THF (15 mL) was added. The reaction mixture was allowed to slowly warm up to room temperature in 2 h, then quenched with 1% AcOH in MeOH solution. Solvent was evaporated and the crude was loaded onto silica gel and purified by flash column (MeOH/DCM 0% to 25% gradient) twice to afford 390 mg (80% pure based on HPLC, detection by 254 nm UV wavelength) of the title compound as off-white solid (Yield 53%).

¹H NMR (400 MHz, DMSO-d₆) δ 1.15-1.05 (m, 6H), 1.20 (d, J=7.20 Hz, 3H), 1.36 (t, J=7.20 Hz, 3H), 4.15-3.75 (m, 4H), 4.55-4.20 (m, 4H), 6.15-6.05 (m, 2H), 6.57 (s, 2H), 7.23 (d, J=8.0 Hz, 2H), 7.42 (d, J=8.0 Hz, 2H), 7.96 (s, 1H).

³¹P NMR (162 MHz, DMSO-d₆) δ 3.93.

MS (m+1)=589.

### Step 3: (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

To a suspension of KO^{t}Bu (0.037 g, 0.33 mmol) in DMSO (4 mL) was added (S)-ethyl 2-(((S)-(4-chlorophenoxy)(((2R,3R,4R,5R)-5-(2,6-diamino-9H-purin-9-yl)-4-fluoro-3-hydroxy-4-methyltetrahydrofuran-2-yl)methoxy)phosphoryl)amino)propanoate (0.15 g, 0.26 mmol). The reaction was stirred at room temperature for 15 min and quenched with 1N HCl solution (˜0.3 mL) at 25° C. to adjust the pH to 4-6. ACN (4 mL) was added to dilute the solution for PREP-HPLC, inorganic salt was filtered before PREP-HPLC. The crude product was directly purified by PREP-HPLC (20% to 70% ACN in 0.1% HCOOH in 8 minutes using XBRIDGE column, flow rate: 20 mL/minute). Desired fast eluting isomer (Rp isomer) was combined and concentrated to dry to afford 22 mg of the title compound (Yield 17%).

¹H NMR (400 MHz, DMSO-d₆) δ 1.10-1.25 (m, 6H), 1.33 (d, J=6.80 Hz, 3H), 3.80 (br, 1H), 4.00-4.30 (m, 3H), 4.45-4.70 (m, 2H), 5.96 (s, 2H), 6.10-6.40 (m, 2H), 6.82 (s, 2H), 7.87 (s, 1H).

³¹P NMR (162 MHz, DMSO-d₆) δ 2.75.

MS (m+1)=461.

### Comparative Example 2

### Preparation of (S)-ethyl 2-(((2R,4aR,6R,7R,7aR)-6-(2,4-dioxo-3,4-dihydropyrimidin-1(2H)-yl)-7-fluoro-7-methyl-2-oxidotetrahydro-4H-furo[3,2-d][1,3,2]dioxaphosphinin-2-yl)amino)propanoate

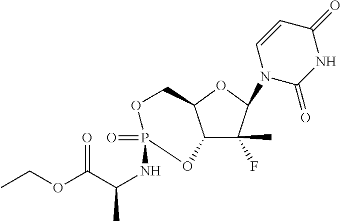 [SMILES: CCOC(=O)[C@H](C)N[P@@]1(=O)OC[C@H]2O[C@@H](n3ccc(=O)[nH]c3=O)[C@](C)(F)[C@@H]2O1]

### Step 1

1-(2R,3R,4R,5R)-3-fluoro-4-hydroxy-5-(hydroxymethyl)-3-methyltetrahydrofuran-2-yl)pyrimidine-2,4(1H,3H)-dione was prepared according to the procedures as described in Sofia, M. J. et al. J. Med. Chem. 2010, 53, 7202-7218.

### Step 2: Synthesis of (2S)-ethyl 2-(((((2R,3R,4R,5R)-5-(2-amino-6-ethoxy-9H-purin-9-yl)-4-fluoro-3-hydroxy-4-methyltetrahydrofuran-2-yl)methoxy)(4-chlorophenoxy)phosphoryl)amino)propanoate

(2R,3R,4R,5R)-5-(2-amino-6-ethoxy-9H-purin-9-yl)-4-fluoro-2-(hydroxymethyl)-4-methyltetrahydrofuran-3-ol (120 mg, 0.46 mmol) was dissolved in THF (7 mL), cooled to 0° C. t-BuMgCl in THF (1.38 mL, 1.38 mmol) was added at 0° C. under argon atmosphere. The reaction mixture was stirred at 0° C. for 30 minutes, and white suspension was formed. (2S)-ethyl 2-(((4-chlorophenoxy)(perfluorophenoxy)phosphoryl) amino)propanoate in THF (3.5 mL) was added to the reaction mixture at 0° C. dropwise. The reaction mixture was allowed to slowly warm up to 25° C. in 2 h, then stirred at 25° C. overnight. The reaction was quenched with saturated NH₄Cl solution (50 mL) at 25° C. and extracted with EA (3×50 mL). The combined organic layer was washed with brine, dried over anhydrous Na₂SO₄, filtered, concentrated, which was then purified by flash column (EA/cyclohexanes 0% to 80% gradient) again to afford 121 mg of the title compound as white solid (Yield 48%).

¹H NMR (400 MHz, DMSO-d₆) δ 1.20-1.11 (m, 3H), 1.30-1.21 (m, 6H), 3.94-3.76 (m, 2H), 4.10-3.95 (m, 4H), 4.43-4.17 (m, 2H), 5.55 (d, J=8.0 Hz, 1H), 5.87-5.80 (m, 1H), 6.18-6.08 (m, 1H), 7.24 (d, J=8.8 Hz, 2H), 7.43 (d, J=8.8 Hz, 2H), 7.58-7.51 (m, 1H).

MS (m+1)=551.

### Step 3: (S)-ethyl 2-((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

A suspension of KO^{t}Bu (49 mg, 0.43 mmol) in DMSO (4 mL) was warmed up to 60° C. for 10 minutes, and then sonicated. At 25° C. was added (2S)-ethyl 2-(((((2R,3R,4R,5R)-5-(2-amino-6-ethoxy-9H-purin-9-yl)-4-fluoro-3-hydroxy-4-methyltetrahydrofuran-2-yl)methoxy)(4-chlorophenoxy)phosphoryl)amino)propanoate (120 mg, 0.22 mmol). The reaction was stirred at room temperature for 10 minutes and quenched with 1N HCl solution at 25° C. to adjust the pH to 4-6, and stirred at room temperature for 10 minutes. ACN (7 mL) was added to dilute the solution for PREP-HPLC, inorganic salt was filtered. The crude product was directly purified by PREP-HPLC (5% to 60% ACN in H₂O in 8 minutes using SUNFIRE column, flow rate: 20 mL/minute). Desired fast eluting isomer (Rp isomer) was combined and concentrated to dry to afford 47 mg of the title compound (Yield 50%).

¹H NMR (400 MHz, DMSO-d₆) δ 1.23 (t, J=7.10 Hz, 3H), 1.25-1.40 (m, 6H), 3.80-3.92 (m, 1H), 4.16 (qd, J=2.00, 7.16 Hz, 2H), 4.20-4.29 (m, 1H), 4.33-4.41 (m, 1H), 4.48-4.67 (m, 2H), 5.79 (d, J=8.04 Hz, 1H), 5.98 (t, J=11.38 Hz, 1H), 6.25 (d, J=21.60 Hz, 1H), 7.55 (d, J=8.04 Hz, 1H), 11.61 (br.s, 1H).

³¹P NMR (162 MHz, DMSO-d₆) δ 3.07.

MS (m+1)=423.

### Comparative Example 3

### Preparation of (2S,4aR,6R,7R,7aR)-6-(2-amino-6-ethoxy-9H-purin-9-yl)-7-fluoro-2-isopropoxy-7-methyltetrahydro-4H-furo[3,2-d][1,3,2]dioxaphosphinine 2-oxide

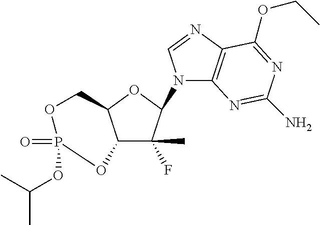 [SMILES: CCOc1nc(N)nc2c1ncn2[C@@H]1O[C@@H]2CO[P@@](=O)(OC(C)C)O[C@H]2[C@@]1(C)F]

### Step 1

(2R,3R,4R,5R)-5-(2-amino-6-ethoxy-9H-purin-9-yl)-4-fluoro-2-(hydroxymethyl)-4-methyltetrahydrofuran-3-ol was prepared according to the procedures (e.g., scheme 6) as described in Reddy, P. G. et al. J. Org. Chem. 2011, 76, 3782-3790.

### Step 2: Synthesis of (2S,4aR,6R,7R,7aR)-6-(2-amino-6-ethoxy-9H-purin-9-yl)-7-fluoro-2-isopropoxy-7-methyltetrahydro-4H-furo[3,2-d][1,3,2]dioxaphosphinine 2-oxide

To a stirred suspension of (2R,3R,4R,5R)-)-5-(2-amino-6-ethoxy-9H-purin-9-yl)-4-fluoro-2-(hydroxymethyl)-4-methyltetrahydrofuran-3-ol (150 mg, 0.46 mmol) in DCM (6 mL) was added triethylamine (0.26 mL, 1.83 mmol) at room temperature and stirred for 10 minutes. The reaction mixture was cooled down to −15° C., then isopropyl phosphorodichloridate (0.14 g, 0.11 mL, 0.78 mmol) was added dropwise via microsyringe. The mixture was stirred at this temperature for 10 minutes and then was added 1-methylimidazole (0.072 mL, 0.92 mmol). The reaction mixture was stirred at −15° C. for 30 minutes, then was allowed to slowly warm up to 0° C. for 2 h. The reaction mixture was diluted with EA (40 mL), washed with 1N HCl soln (2×15 mL) and brine. The EA layer was concentrated, diluted with MeOH, purified by PREP-HPLC directly (5% to 95% ACN in 0.1% HCOOH in 8 minutes using XBRIDGE column, flow rate: 20 mL/minute) to give 124 mg of the title compound as white solids (Yield 63%).

¹H NMR (400 MHz, DMSO-d₆) δ 1.36 (d, J=24.0 Hz, 3H), 1.43-1.53 (m, 9H), 4.30-4.70 (m, 5H), 4.80-4.95 (m, 1H), 5.05 (br, 2H), 5.36 (br, 1H), 6.06 (d, J=19.32 Hz, 1H), 7.67 (s, 1H).

³¹P NMR (162 MHz, CHLOROFORM-d) δ −7.16.

MS (m+1)=432.

### Comparative Example 4

### Preparation of (S)-isopropyl 2-(((S)-(((2R,3R,4R,5R)-5-(2-amino-6-methoxy-9H-purin-9-yl)-4-fluoro-3-hydroxy-4-methyltetrahydrofuran-2-yl)methoxy) (phenoxy)phosphoryl)amino)propanoate

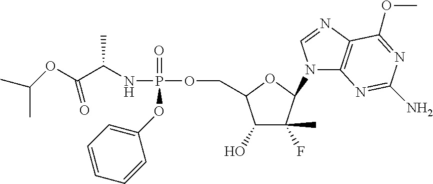 [SMILES: COc1nc(N)nc2c1ncn2[C@@H]1OC(CO[P@@](=O)(N[C@@H](C)C(=O)OC(C)C)Oc2ccccc2)[C@@H](O)[C@@]1(C)F]

(2R,3R,4R,5R)-5-(2-amino-6-methoxy-9H-purin-9-yl)-4-fluoro-2-(hydroxymethyl)-4-methyltetrahydrofuran-3-ol was prepared according to the procedures (e.g., scheme 6) as described in Reddy, P. G. et al. J. Org. Chem. 2011, 76, 3782-3790.) (100 mg, 0.319 mmol) was dissolved in THF (4 mL), cooled to −20° C. t-BuMgCl in THF (1.0 M, 1.28 mL, 1.28 mmol) was added at −20° C. under argon atmosphere, a clear solution formed. The reaction mixture was stirred at −20° C. for 120 minutes.

(S)-isopropyl-2-((R)-(perfluorophenoxy)(phenoxy)phosphoryl)amino)propanoate (434 mg, 0.957 mmol) in THF (4 mL) was added to the reaction mixture at −20° C. under argon atmosphere and stirred for 2 h 30 minutes. The reaction was allowed to warmed up to room temperature and concentrated. Methanol (2 mL) was added to dilute the solution for PREP-HPLC, inorganic salt was filtered. The crude product was directly purified by PREP-HPLC (40% to 95% methanol in H₂O with 0.1% formic acid in 40 minutes using Atlantis C18 column, flow rate 20 mL/minute). Desired slow eluting isomer was combined and concentrated to dry to afford 28.3 mg of the title compound as white solid (Yield 15%).

¹H NMR (400 MHz, DMSO-d₆) δ 1.24-1.02 (m, 12H), 3.72-3.87 (m, 1H), 3.97 (s, 3H), 4.09 (td, J=6.9, 5.6, 3.8 Hz, 1H), 4.25-4.46 (m, 3H), 4.80 (hept, J=6.4 Hz, 1H), 5.79 (d, J=7.3 Hz, 1H), 5.98 (dd, J=12.9, 10.0 Hz, 1H), 6.61 (s, 2H), 6.10 (d, J=19.2 Hz, 1H), 7.13-7.26 (m, 3H), 7.30-7.40 (m, 2H), 7.98 (s, 1H).

³¹P NMR (162 MHz, DMSO-d₄) δ 3.80.

MS (m+1)=584.

### Pharmacological Data

The utility of the compounds of the present invention may be evidenced by using any one of the assays described herein below.

### Dengue EC₅₀ Determination

Cryopreserved PBMC (Peripheral Blood Mononuclear Cell) cells were purchased from AllCells, LLC in USA. The cells were then thawed according to manufacturer's instructions and re-suspended in RPMI medium supplemented with 1% penicillin/streptomycin solution and 10% Fetal Calf serum. The cells were then counted and viability checked (viability should be at least 80%). After centrifuging and removing the media, the cells were diluted to 1×10⁷ cells/mL in RPMI medium supplemented with 1% penicillin/streptomycin. 50 μl of the cells were then dispensed into 96-well tissue culture plate resulting in 5×10⁵ cells/well. Next, virus with humanized 4G2 mixture was prepared for infection. Briefly, virus (2×10⁷ pfu/ml) was mixed with humanized 4G2 antibody with the final antibody concentration of 0.38 μg/ml and incubated for 30 minutes at 4° C. to assist virus/antibody complex formation. The virus-antibody complex was then added to the PBMC at multiplicity of infection (M.O.I) of 1. The mixture of cells, virus and antibody was then further incubated in the plates at 37° C. for an hour in the humidified incubator for virus attachment and infection to take place. Serial diluted compounds were then added to the final media volume of 200 μl with 2% Fetal Calf Serum (final PBMC concentration would be 2.5×10⁶ cells/mL). The plates were then incubated at 37° C., 5% CO₂ for another 48 hours. The extent of the infection and compound inhibition was measured by plaque reduction assay using BHK cells [RD-2004-80036]. Briefly, supernatants of BHK cells grown in 24-well tissue culture (seeded at 200,000 cells/well the night before) were removed and subjected to 200 μl of diluted supernatants derived from PBMC infection containing serial diluted compounds. After incubating it for 37° C. for 1 hour in the incubator, the supernatants were removed and BHK cells overlaid with RPMI media containing 0.8% methyl-cellulose, 0.5% DMSO, 0.05% sodium bicarbonate, 25 mM HEPES, 2% fetal bovine serum and 1% penicillin/streptomycin solution. After additional 4 days of incubation at 37° C. incubator, the monolayer of BHK cells were fixed with 4% paraformaldehyde, stained with crystal violet and plaques counted. The dose response (n=2) is calculated by Prism from Graphpad software using a mathematical curve fitting analysis called nonlinear regression with four independent parameters. Briefly, the software generates the best fit curve using the formula F(x)=((A−D)/(1+((x/C)̂B)))+D. In this formula, x represents the concentration of the compound, A is the minimum value, B represents the steepness of the curve (sometimes known as Hill slope), C is the inflection point and D is the maximum value. EC₅₀ was estimated as the concentration of the compound which will inhibit 50% of the plaque formation. A positive control (7-deaza-2′-C-acetylene-adenosine) was used to ensure the quality of the data.

Dengue EC₅₀ Data of some representative compounds are shown in Table 4 below.

TABLE 4

| Example # | Compound | Dengue EC₅₀ (μM) |
|---|---|---|
| 3 | 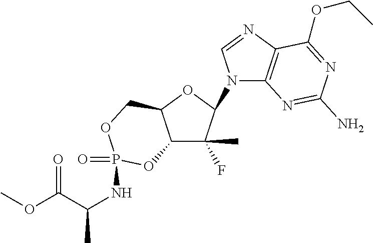 [SMILES: CCOc1nc(N)nc2c1ncn2[C@@H]1O[C@@H]2CO[P@](=O)(N[C@@H](C)C(=O)OC)O[C@H]2[C@@]1(C)F] | 0.46 |
| 1 | 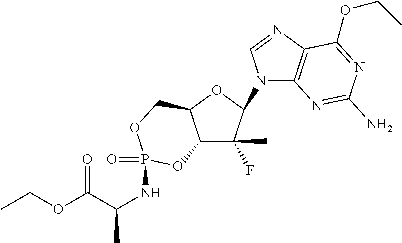 [SMILES: CCOC(=O)[C@H](C)N[P@@]1(=O)OC[C@H]2O[C@@H](n3cnc4c(OCC)nc(N)nc43)[C@](C)(F)[C@@H]2O1] | 0.23 |
| 2 | 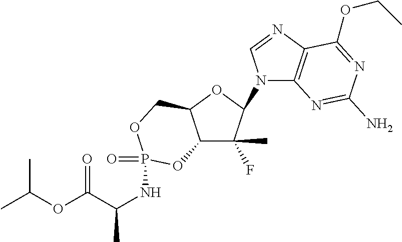 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F] | 0.072 |
| 6 | 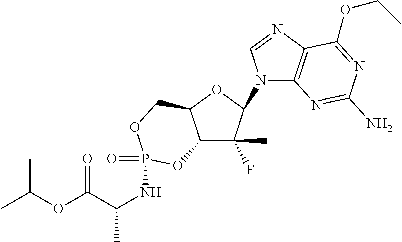 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | 0.37 |
| 8 | 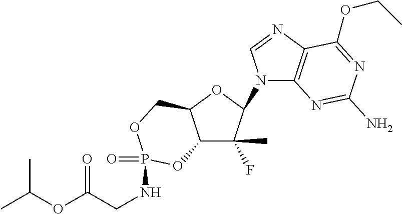 [SMILES: CCOc1nc(N)nc2c1ncn2[C@@H]1O[C@@H]2CO[P@](=O)(NCC(=O)OC(C)C)O[C@H]2[C@@]1(C)F] | 0.33 |
| 11 | 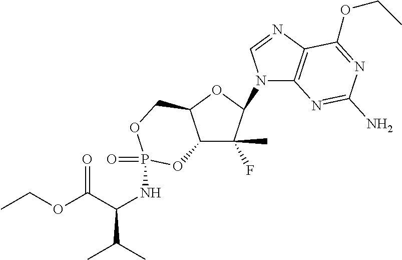 [SMILES: CCOC(=O)[C@@H](N[P@]1(=O)OC[C@H]2O[C@@H](n3cnc4c(OCC)nc(N)nc43)[C@](C)(F)[C@@H]2O1)C(C)C] | 0.23 |
| 12 | 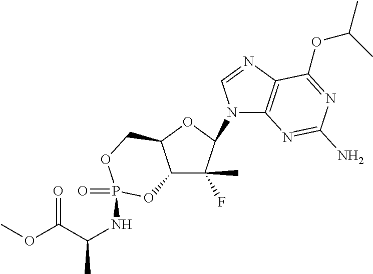 [SMILES: COC(=O)[C@H](C)N[P@@]1(=O)OC[C@H]2O[C@@H](n3cnc4c(OC(C)C)nc(N)nc43)[C@](C)(F)[C@@H]2O1] | 0.43 |
| 14 | 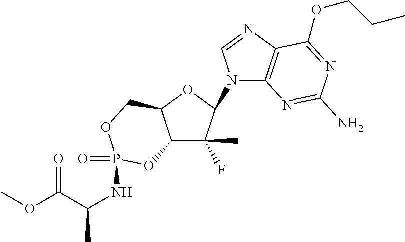 [SMILES: CCCOc1nc(N)nc2c1ncn2[C@@H]1O[C@@H]2CO[P@](=O)(N[C@@H](C)C(=O)OC)O[C@H]2[C@@]1(C)F] | 0.18 |
| Comparative Example # 1 | 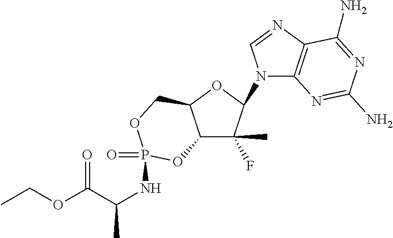 [SMILES: CCOC(=O)[C@H](C)N[P@@]1(=O)OC[C@H]2O[C@@H](n3cnc4c(N)nc(N)nc43)[C@](C)(F)[C@@H]2O1] (comparator) | >25 |
| Comparative Example # 2 | 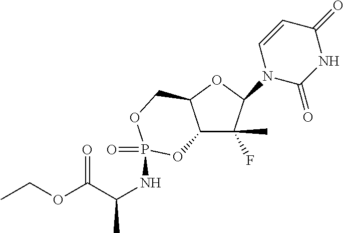 [SMILES: CCOC(=O)[C@H](C)N[P@@]1(=O)OC[C@H]2O[C@@H](n3ccc(=O)[nH]c3=O)[C@](C)(F)[C@@H]2O1] (comparator) | >25 |
| Comparative Example # 3 | 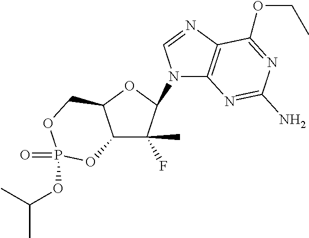 [SMILES: CCOc1nc(N)nc2c1ncn2[C@@H]1O[C@@H]2CO[P@@](=O)(OC(C)C)O[C@H]2[C@@]1(C)F] (comparator) | >25 |

### Liver S9 Stability Determination

The liver is the main organ of metabolism and contains a high concentration of drug metabolizing enzymes. Metabolic clearance, particularly hepatic, is one of the main determinants affecting systemic drug exposure following both oral and i.v. administration. To assess the ability of the compounds of the invention to reach a systemic circulation, the hepatic S9 fraction was chosen to test for stability of the compounds. Hepatic S9 fraction was obtained following centrifugation of homogenized liver tissue at 9,000 g. The hepatic S9 fraction is composed of both microsomal and cytosolic fractions and contains all the enzymes contained in microsomes plus additional phase I and phase II cytosolic enzymes.

Stock solutions of each tested compound (100 μM) were prepared in methanol by two subsequent 1:10 dilutions from 10 mM DMSO drug solutions, the final assay concentration was 1 μM. Frozen pooled liver S9 fraction was thawed from −20° C. and diluted in phosphate buffer (with and without NADPH) which was pre-warmed at 37° C. for 10 minutes. The reaction was initiated by addition of compound. Manual sample plates were incubated on a Thermo Max Q 2000 shaker placed in a temperature controlled Sartorius Certomat H hood set at 37° C. Sequential samples were removed at designated timepoints (0, 5, 15, 30, 60 and 120 mins) and quenched with 4 volumes of ice cold acetonitrile (containing internal standard, diazepam), centrifuged and supernatants reconstituted in water (acetonitrile:water, 50:50% v/v). Samples were then analyzed by LCMS/MS or LC-UV to assess parent depletion. In vitro half-life (t_{1/2}) is calculated based on the rate of disappearance of compound from the reaction mixture at incubation timepoints up to 2 hours.

Stability of some representative compounds in human liver S9 fraction and liver microsomes are shown in Table 5 below. The data shows compounds of the present invention are stable under the condition of the liver.

TABLE 5

| | | liver S9 | liver |
|---|---|---|---|
| | | t_{1/2} | microsomes |
| Example # | Compound | (minute) | t_{1/2} (minute) |
| 3 | 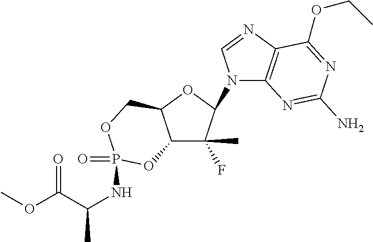 [SMILES: CCOc1nc(N)nc2c1ncn2[C@@H]1O[C@@H]2CO[P@](=O)(N[C@@H](C)C(=O)OC)O[C@H]2[C@@]1(C)F] | >120 | 57 |
| 1 | 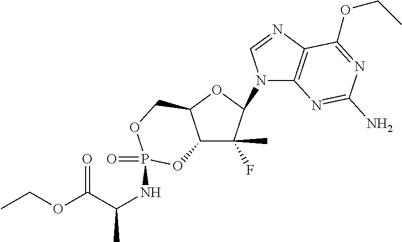 [SMILES: CCOC(=O)[C@H](C)N[P@@]1(=O)OC[C@H]2O[C@@H](n3cnc4c(OCC)nc(N)nc43)[C@](C)(F)[C@@H]2O1] | 76 | 47 |
| 2 | 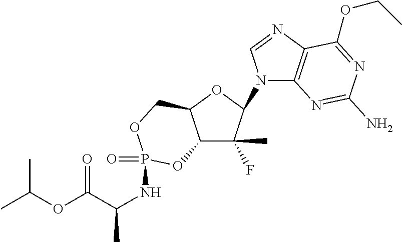 [SMILES: CCOc1nc(N)nc2c1ncn2[C@@H]1O[C@@H]2CO[P@](=O)(N[C@@H](C)C(=O)OC(C)C)O[C@H]2[C@@]1(C)F] | 36 | 32 |
| 6 | 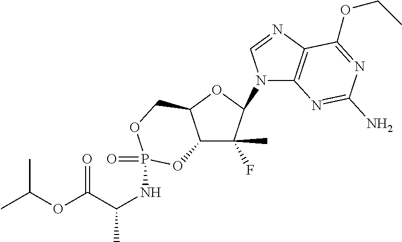 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | >120 | |
| 8 | 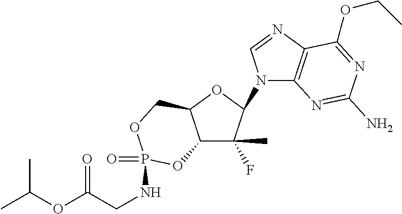 [SMILES: CCOc1nc(N)nc2c1ncn2[C@@H]1O[C@@H]2CO[P@](=O)(NCC(=O)OC(C)C)O[C@H]2[C@@]1(C)F] | 57.5 | |
| 11 | 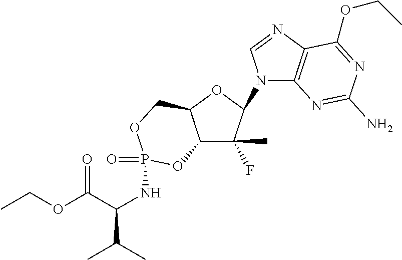 [SMILES: CCOC(=O)[C@@H](N[P@]1(=O)OC[C@H]2O[C@@H](n3cnc4c(OCC)nc(N)nc43)[C@](C)(F)[C@@H]2O1)C(C)C] | 82 | |
| Comparative Example # 4 | 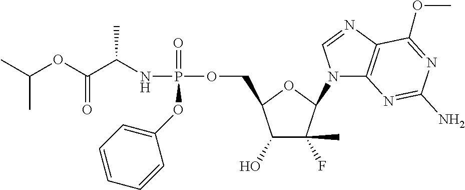 [SMILES: COc1nc(N)nc2c1ncn2[C@@H]1O[C@H](CO[P@@](=O)(N[C@@H](C)C(=O)OC(C)C)Oc2ccccc2)[C@@H](O)[C@@]1(C)F] (comparator) | 18 | 6 |

### Cytotoxicity Determination

The cytotoxicity of compounds of the invention can be determined using the following general protocol.

### CCK8 Cytotoxicity Assay in HepG2 Cells

HepG2 cells were trypsinized, washed, counted and diluted to 1.6×10⁴ cells/ml in DMEM-W/O Glucose, supplemented with 10% Fetal bovine serum (FBS), 1% Penicillin/Streptomycin, 2 mM HEPES, 1 mM Sodium Pyruvate, 10 mM Galactose & 2 mM Glutamine. 25 μl of the media containing 400 cells per well were dispensed in clear 384-well tissue culture plate and incubated at room temperature for 30 minutes. The plate was then transferred and placed at 37° C., 5% CO₂ humidified incubator overnight. On the next day, serial-diluted compound plates were prepared and 125 nl of compounds at various concentrations were then dispensed into the tissue culture well (200× dilution). The plates were then transferred to 37° C., 5% CO₂ humidified incubator for additional 96 hours. Cytotoxicity was measured by CCK-8 assay. Briefly, CCK-8 was thawed on bench top and diluted 2.5× with the growth media. 35 ul of the pre-diluted CCK-8 was then introduced into each well and the plates were then further incubated in 37° C., 5% CO₂ humidified incubator for 3 hours. The absorbance was read by Envision at 450 nm. Dose response curves were calculated as in the previous section. The CC₅₀ is estimated as the concentration of the compound which will inhibit 50% of the signal. A positive control (puromycin) was used to ensure the quality of the data.

### CCK8 Cytotoxicity Assay in THP-1 Cells

4 day cytotoxicity assay using THP-1 cells: THP-1 cells grown in suspension were counted and diluted to 8×10⁴ cells/ml in RPMI-1640 media supplemented with 10% fetal bovine serum (FBS) and 1% penicillin/streptomycin. 25 ul of the THP-1 containing media consisting of 2000 cells were dispensed in 384-well tissue culture plate and pre-incubated at room temperature for 30 minutes, followed by 37° C., 5% CO₂ overnight in the humidified incubator. On the next day, serial-diluted compound plates were prepared and 125 nl of compounds at various concentrations were then dispensed into the tissue culture well (200× dilution). The plates were then transferred to 37° C., 5% CO₂ humidified incubator for additional 96 hours. The plates were then transferred to 37° C., 5% CO₂ humidified incubator for additional 96 hours. Cytotoxicity was measured by CCK-8 assay. Briefly, CCK-8 was thawed on bench top and diluted 2.5× with the growth media. 35 ul of the pre-diluted CCK-8 was then introduced into each well and the plates were then further incubated in 37° C., 5% CO₂ humidified incubator for 3 hours. The absorbance was read by Envision at 450 nm. Dose response curves were calculated as in the previous section (n=2). The CC₅₀ is estimated as the concentration of the compound which will inhibit 50% of the signal. A positive control (puromycin) was used to ensure the quality of the data.

Cytotoxicity Data of some representative compounds are shown in Table 6 below. The data (together with the data in Table 4) indicates that compounds of the present invention have good selectivity, because while they are active against cells infected by dengue virus, they are not toxic to normal cells.

TABLE 6

| | | HepG2 | THP-1 |
|---|---|---|---|
| Example # | Compound | CC₅₀ (μM) | CC₅₀ (μM) |
| 3 | 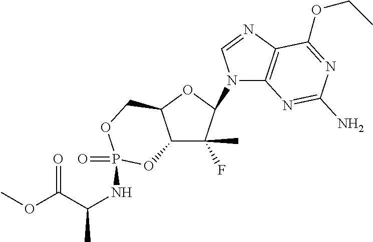 [SMILES: CCOc1nc(N)nc2c1ncn2[C@@H]1O[C@@H]2CO[P@](=O)(N[C@@H](C)C(=O)OC)O[C@H]2[C@@]1(C)F] | >50 | >50 |
| 1 | 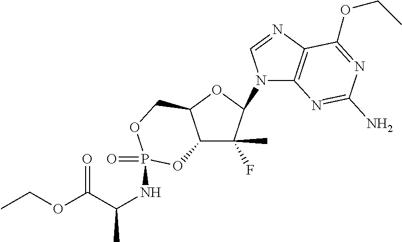 [SMILES: CCOC(=O)[C@H](C)N[P@@]1(=O)OC[C@H]2O[C@@H](n3cnc4c(OCC)nc(N)nc43)[C@](C)(F)[C@@H]2O1] | >50 | >50 |
| 2 | 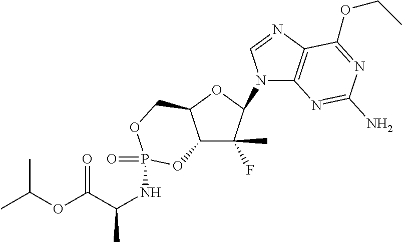 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F] | >50 | 34 |
| 6 | 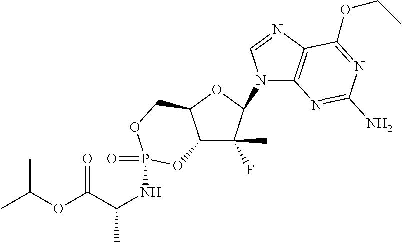 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | >50 | >50 |
| 8 | 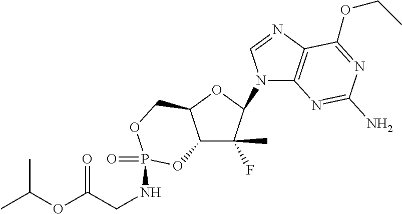 [SMILES: CCOc1nc(N)nc2c1ncn2[C@@H]1O[C@@H]2CO[P@](=O)(NCC(=O)OC(C)C)O[C@H]2[C@@]1(C)F] | >50 | >50 |
| 11 | 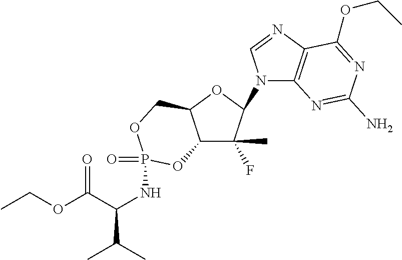 [SMILES: CCOC(=O)[C@@H](N[P@]1(=O)OC[C@H]2O[C@@H](n3cnc4c(OCC)nc(N)nc43)[C@](C)(F)[C@@H]2O1)C(C)C] | >50 | >50 |
| 12 | 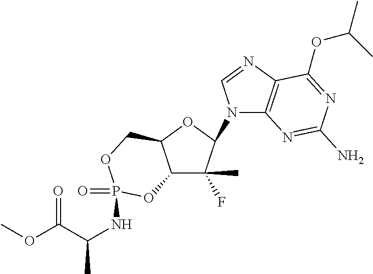 [SMILES: COC(=O)[C@H](C)N[P@@]1(=O)OC[C@H]2O[C@@H](n3cnc4c(OC(C)C)nc(N)nc43)[C@](C)(F)[C@@H]2O1] | >50 | >50 |
| 14 | 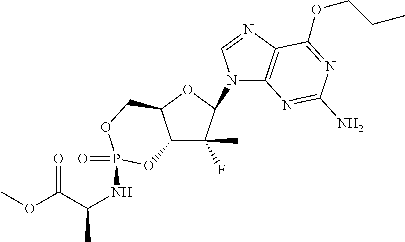 [SMILES: CCCOc1nc(N)nc2c1ncn2[C@@H]1O[C@@H]2CO[P@](=O)(N[C@@H](C)C(=O)OC)O[C@H]2[C@@]1(C)F] | >50 | >50 |

Determination of the Intracellular Nucleoside Triphosphate Concentration Needed to Inhibit 50% of Viral Production (NTP₅₀)

Like any other viral polymerases, flaviviral polymerase including HCV and Dengue is responsible for generating the viral genome for propagation and viral protein synthesis. All flaviviral polymerase are classified as RNA dependent RNA polymerase (RdRp) where the virus uses RNA as the template and ribose nucleoside triphosphates as substrates. Inhibition of viral replication by nucleosides has been extensively studied (De Clercq, E. (2001) J. Clin. Virol. 22:73-89) including nucleosides that inhibit RdRp. Generally, the antiviral activity of these nucleosides are attributed to the conversion of the nucleosides to nucleoside 5′-monophosphate (NMP), and then from NMP to the corresponding nucleoside triphosphate (NTP) where the NTP acts as inhibitors of DNA and RNA polymerases or as chain terminators following incorporation into the lengthening viral DNA or RNA strand.

The conversion from nucleoside to NMP (by the nucleoside kinase) is generally viewed as the rate limiting step of the three phosphorylation events. To circumvent the need for the initial phosphorylation step in the metabolism of a nucleoside to the active NTP analog, NMP prodrugs have been used to bypass the poor nucleoside kinase activity (Schultz, Bioorg. Med. Chem. 2003, 11, 885). Once the NMP prodrugs reach into a target organ or cell, they will be cleaved to the NMP and subsequently converted to NTP. Therefore, the intracellular concentration of nucleoside triphosphates (NTPs) derived from the NMP prodrugs would provide the most direct measurement to the efficacy of the NMP prodrugs.

NTP₅₀ is defined as the concentration of the nucleoside triphosphate (NTP) that will inhibit 50% of viral production. The NTP₅₀ experiment was performed using cryopreserved PBMC as described above in the Dengue EC₅₀ determination section. Briefly, PBMC was thawed and incubated in RPMI medium containing 2% Fetal Bovine serum (FBS), 1% Penicillin/Streptomycin supplemented with various concentration of a representative compound of the present invention (e.g., Example 2) up to 100 μM. After the intracellular conversion of Example 2 into corresponding nucleoside triphosphate reached steady state (24 hours at 37° C. in the incubator), the cells were spun down, washed with 0.9% NaCl solution and lyzed in RIPA buffer (50 mM Tris pH 7.5, 150 mM NaCl, 1% NP40) containing with protease and phosphotase inhibitor. The cells were further incubated at room temperature for complete lysis of the cells and the cell debris spun down at 13,000 g in at 4° C. for 20 minutes. The lysate was transferred into cells and the concentration of the nucleoside triphosphate was measured. The concentration of the nucleoside triphosphate was then plotted against the concentration of Example 2 and a curve of the concentration of the nucleoside triphosphate vs incubated Example 2 was generated using Michaelis-Menten Kinetics with the formula Y=A[Example 2]/B+[Example 2] where Y is the concentration of the nucleoside triphosphate and [Example 2] is the concentration of the compound Example 2. A and B were constants determined by best fitting. By EC₅₀ of [Example 2] in the equation, the intracellular concentration of nucleoside triphosphate which corresponds to 50% reduction of the virus (NTP₅₀) can be determined.

Since all compounds of the present invention would be converted to the same nucleoside triphosphate intracellularly (the structure of NTP is shown below), the NTP₅₀ of all compounds of the present invention would be the same regardless which compound was used in the experiment. NTP₅₀ for the compound Example 2 was measured to be 0.0016 μM in PBMC lysate from 1 million cells with 100 μL of lysis buffer. The Mean Corpuscular Volume of PBMC is approximately 0.3×10⁻⁶ μL/per cell=0.3 μL/1 million cells. By using the literature of Simiele, M. et al. Antimicrob. Agents Chemother. 2011 55, 2976-2978, NTP₅₀=0.0016×(100/0.3)=0.53 μM/1 million cells=0.53×0.3/1.0×10⁶=0.159×10⁻⁶ μmol/1 million cells. Thus, from the calculation, the intracellular nucleoside triphosphate concentration needed to inhibit 50% of viral production (NTP₅₀) is around ˜200 fmol/1 million cells.

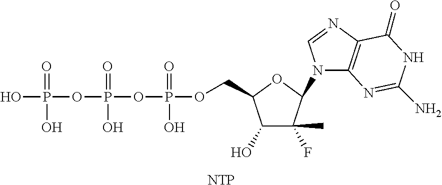 [SMILES: C[C@@]1(F)[C@H](O)[C@@H](COP(=O)(O)OP(=O)(O)OP(=O)(O)O)O[C@H]1n1cnc2c(=O)[nH]c(N)nc21 ; "NTP"]

### Determination of Nucleoside Triphosphate Concentrations (“NTP”) in DOG PBMC Cells

Nucleoside Triphosphate Concentrations derived from the compounds of the present invention in dog PBMC cells were measured according to the following experimental procedures.

### Pharmacokinetics

Male beagle dogs were administered a single intravenous (0.5 mg/kg) or oral dose (3-15 mg/kg) of a representative compound of the present invention. Blood samples (3.5 mL) were collected at 0, 0.0833 (IV only), 0.25, 0.5, 1, 2, 4, 6, 8, 24, 48, 72 hours post-dose through the jugular vein. K₂EDTA was used as the anticoagulant. Plasma was prepared from an aliquot of 0.5 mL of blood and assayed for the compound and metabolites by tandem mass spectrometric method (LC/MS-MS). PBMC was isolated from 3 mL of blood and assayed for nucleoside triphosphate (NTP) levels by LC/MS-MS.

### Isolation of Whole Blood Dog Pbmc

Three mL of blood was collected from the dog into EDTA vacutainers and mixed on a rocker & immediately placed on ice. The blood was transferred to a 15 mL falcon tube. Three mL PBS (w/o Ca/Mg) and 3 mL 6% dextran (in PBS) were added and was left for 30 minutes on ice to allow the red blood cells to sediment. Blood supernatant (5-7 mL) was layered on top of 5 mL of Mammalian-Lympholyte in 15-mL conical blue-top tube, centrifuged for 20 minutes at 1500 g at 4° C.

After centrifugation, the well-defined lymphocyte (PBMC) layer at the interface was carefully removed to a new 15-mL centrifuge tube and was diluted to a total volume of 15 mL to reduce the density of the solution. The content was centrifuged at 800 g for 10 minutes at 4° C. to pellet the PBMC. The pellet was re-suspended in 1 mL of PBS and further diluted to 10 mL, followed by centrifugation at 800 g for 10 minutes at 4° C. to pellet the PBMC. The pellet was re-suspended in 1 mL PBS and followed by 5 mL ice cold de-ionized water, mixed for 30 seconds by inverting to lyse the remaining red blood cells. Five ml of ice cold 2×PBS was added to bring the mixture back to isotonicity. The mixture was centrifuged at 800 g for 10 minutes at 4° C. to pellet the cells and discard the supernatant. The resulting pellet was re-suspended in 1 mL PBS. An aliquot of 10 μL of the pellet suspension was diluted into 90 μL of PBS for cell count. An aliquot of each sample was transferred to a fresh Eppendorf tubes and centrifuged at 5000 rpm for 5 min at 4° C. The pellets were ready for bioanalysis.

### Bioanalysis

Concentrations of nucleoside triphosphate in PBMC were determined by tandem mass spectrometric method (LC/MS/MS). Chromatography was conducted on ACQUITY UPLC HSS T3 Column, 100 Å, 1.8 μm, 2.1 mm×50 mm using a mobile phase gradient consisting of (A) 2 mM ammonium dihydrogen-phosphate and 5 mM Hexylamine in (B) Acetonitrile, 8% B to 22% B in 3.2 minutes at a flow rate of 0.3 mL/minute. The mass spectrometer was operated in the Multiple Reaction Monitoring (MRM) using negative ionization mode. An internal standard, Carbosynth was used in the construction of standard calibration curves in blank PBMC obtained commercially. The nucleoside triphosphate was monitored at m/z 538.1>158.9 and the internal standard was monitored at 536.1>158.9. The dynamic range was 50, 100, 300, 1000, 3000, 10000, 30000 fmole/3×10⁶ PBMC.

Nucleoside Triphosphate Concentrations of some representative compounds of the present invention (after oral administration of 3 mg/Kg of the compounds) are shown in Table 7 below.

TABLE 7

| | | NTP Cₘₐₓ | NTP AUC₀₋₂₄ₕ |
|---|---|---|---|
| | | (fmol/ | (fmol/ |
| | | 3 million | 3 million |
| Example # | Compound | cells) | cells) |
| 3 | 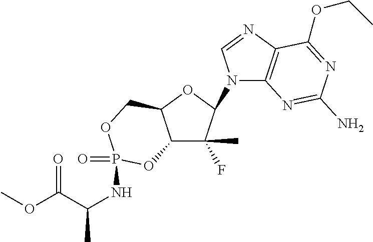 [SMILES: CCOc1nc(N)nc2c1ncn2[C@@H]1O[C@@H]2CO[P@](=O)(N[C@@H](C)C(=O)OC)O[C@H]2[C@@]1(C)F] | 662 ± 228 | 10457 ± 2983 |
| 1 | 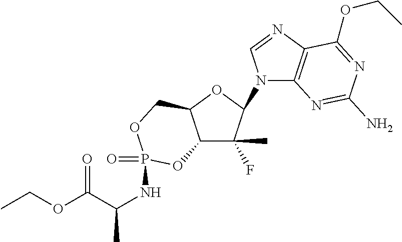 [SMILES: CCOC(=O)[C@H](C)N[P@@]1(=O)OC[C@H]2O[C@@H](n3cnc4c(OCC)nc(N)nc43)[C@](C)(F)[C@@H]2O1] | 2331 ± 1181 | 36900 ± 11358 |
| 2 | 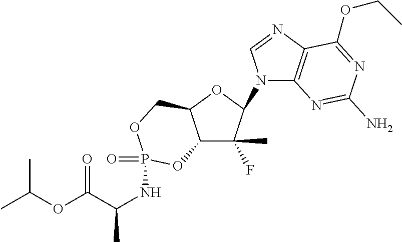 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F] | 618 ± 308 | 7218 ± 5117 |
| 6 | 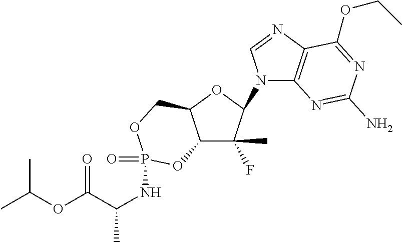 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | 777 ± 45.2 | 11049 ± 2190 |

As a blood-borne disease, PBMC (Peripheral Blood Mononuclear Cell), is believed to be one of the major sites for dengue virus replication. Therefore, achieving high concentration of nucleoside triphosphate in PMBC is desirable to inhibit dengue virus replication. The above data in Table 7 demonstrate that compounds of the present invention, after orally administration at the dosage of 3 mg/kg, have the advantage of producing high concentrations of the nucleoside triphosphate (e.g., larger than the NTP₅₀), which offers particular advantage for treatment of systemic viral infections, especially dengue.

### Summary of the Advantages of the Compounds of the Present Invention

The poor conversion of the nucleoside to NTP can often be attributed to the inability of nucleoside kinases to convert the nucleoside to the nucleoside 5′-monophosphate (NMP). NMP prodrugs have been used to bypass poor nucleoside kinase activity. However, the existing NMP prodrugs in the art suffer several drawbacks. For example, many NMP prodrugs cause substantial toxicity because the corresponding NTP lacks adequate specificity for viral polymerases compared to host polymerases and. In addition, many NMP prodrugs have poor physiochemical and pharmacokinetic properties, which limit their absorption and uptake into the target tissue or cell. For instance, some NMP prodrugs are substrates for metabolizing enzymes in the liver, esterases and phosphodiesterases in the blood and other body tissues, which can cleave the prodrug to a charged molecule or to the nucleoside, respectively. The charged molecule is then impermeable to the target organ or cell and the nucleoside is poorly phosphorylated intracellularly.

The development of a non-toxic, highly effective and bioavailable NMP prodrug is largely an unpredictable trial and error exercise requiring the balancing of the stability of the NMP prodrug in various body organs/tissues (e.g., GI tract, liver, etc.) and blood with the ability of the prodrug to reach a target organ or cell, be absorbed or actively taken up by the target cell, being efficiently cleaved to the NMP intracellularly and subsequently converted to a NTP that is selective for inhibiting the viral polymerase. It has been surprisingly found that the compounds of the present invention demonstrate superior balanced profile over the NMP prodrugs in the art. The compounds of the present invention have improved stability under the condition of liver and effectively evade first pass metabolism, thereby deliver a wider distribution of higher levels of NTP which is selective for inhibiting the viral polymerase.

### Polymorphic forms of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

### Example 16

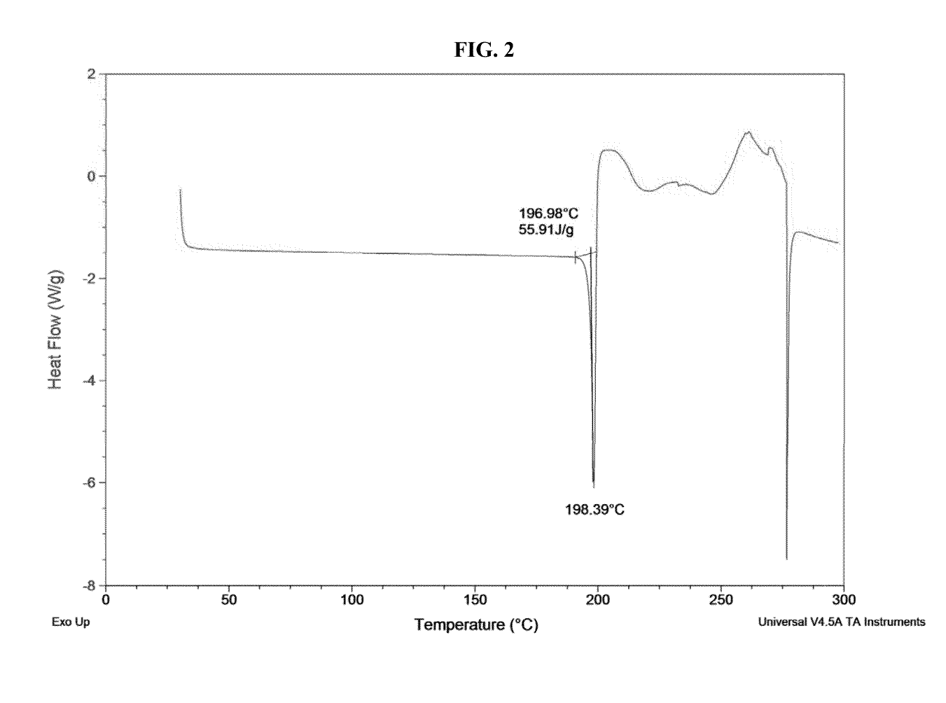

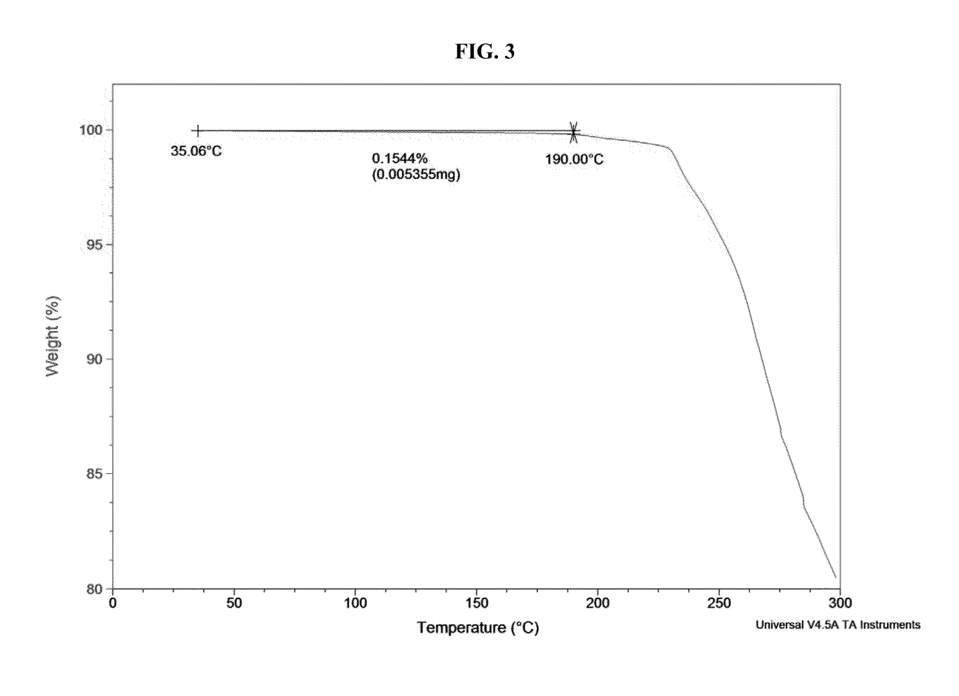

The characteristic 2-theta (2θ) values, d-spacing (A) and relative intensity (RI) for the powder X-ray diffraction (PXRD) pattern of the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form I”) are shown in Table 8 (below). The corresponding DSC and TGA thermogram for the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form I”) is shown in FIG. 2 and FIG. 3, respectively.

TABLE 8

| 2-Theta | d-spacing | Relative |
|---|---|---|
| (deg°) | (Å) | Intensity (%) |
| 7.6 | 11.66 | high |
| 10.3 | 8.55 | low |
| 11.1 | 7.94 | high |
| 11.8 | 7.52 | medium |
| 12.3 | 7.18 | low |
| 15.2 | 5.84 | low |
| 16.5 | 5.38 | low |
| 18.1 | 4.90 | low |
| 19.1 | 4.64 | medium |
| 19.9 | 4.46 | low |
| 20.7 | 4.28 | medium |
| 21.5 | 4.13 | low |
| 22.2 | 4.00 | low |
| 23.6 | 3.77 | low |
| 25.3 | 3.52 | low |
| 29.5 | 3.03 | low |

### Example 17

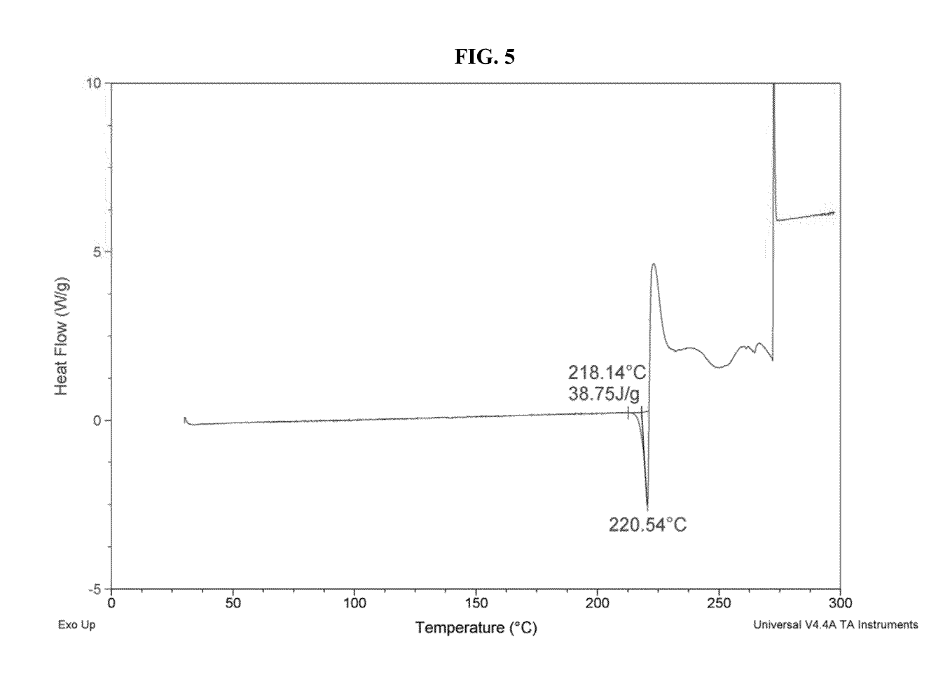

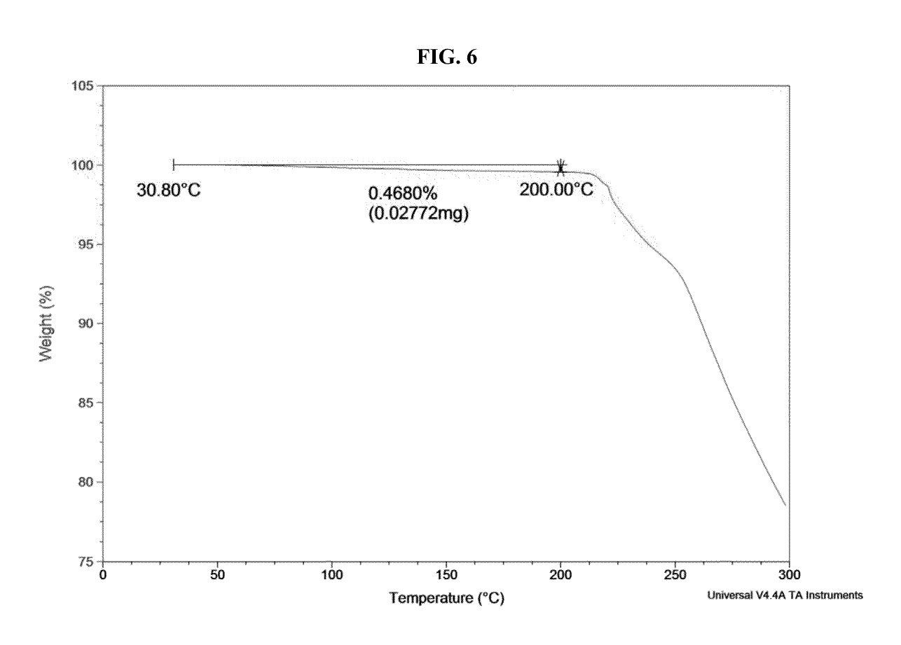

The characteristic 2-theta (2θ) values, d-spacing (Å) and relative intensity (RI) for the powder X-ray diffraction (PXRD) pattern of the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form II”) are shown in Table 9 (below). The corresponding DSC and TGA thermogram for the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form II”) is shown in FIG. 5 and FIG. 6, respectively.

TABLE 9

| 2-Theta | d-spacing | Relative |
|---|---|---|
| (deg°) | (Å) | intensity |
| 8.1 | 10.95 | low |
| 10.8 | 8.18 | low |
| 11.4 | 7.73 | high |
| 12.2 | 7.27 | medium |
| 12.7 | 6.98 | medium |
| 14.5 | 6.12 | low |
| 15.6 | 5.67 | medium |
| 18.1 | 4.92 | low |
| 19.1 | 4.64 | low |
| 20.1 | 4.42 | low |
| 20.3 | 4.37 | low |
| 21.7 | 4.09 | medium |
| 22.7 | 3.92 | low |
| 23.0 | 3.87 | low |
| 23.7 | 3.75 | low |
| 24.4 | 3.65 | low |
| 25.3 | 3.52 | high |
| 25.7 | 3.47 | high |
| 27.2 | 3.27 | low |

### Example 18

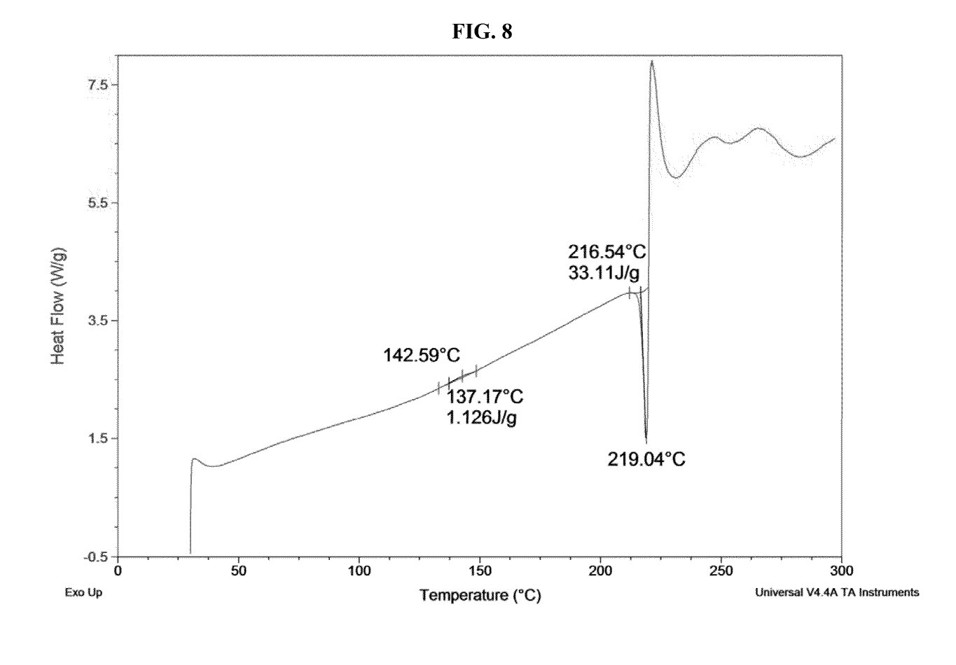

The characteristic 2-theta (2θ) values, d-spacing (A) and relative intensity (RI) for the powder X-ray diffraction (PXRD) pattern of the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form III”) are shown in Table 10 (below). The corresponding DSC thermogram for the crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate (“Form III”) is shown in FIG. 8.

TABLE 10

| 2-Theta | d-spacing | Relative |
|---|---|---|
| (deg°) | (Å) | intensity |
| 7.7 | 11.43 | high |
| 12.3 | 7.22 | high |
| 15.5 | 5.72 | low |
| 16.6 | 5.34 | medium |
| 17.4 | 5.08 | low |
| 20.0 | 4.44 | low |
| 22.1 | 4.02 | low |
| 22.9 | 3.88 | medium |
| 24.6 | 3.62 | medium |
| 35.6 | 2.53 | low |

The polymorphic forms of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate were prepared as described below and were evaluated using the following instrumentation and procedures.

Differential Scanning Calorimetry (DSC):

TA Instruments: DSC Q2000 Version 2.8.0.392 and Processing software: Universal Analysis 2000 Version 4.4A TA instrument. Temperature increase rate=10° C./minute with a range of 30° C. to 300° C.

Thermogravimetric Analysis (TGA):

TA Instruments; TGA Q5000 Version 2.8.0.392 and Processing Software: Universal Analysis 2000 Version 4.4A TA instrument.

X-Ray Powder Diffraction (XRPD):

Bruker D8 Discover, Madison, Wis., USA

### Preparation of “Form I” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

2.0 mg of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate obtained according to Example 1, was dissolved in 150 μL of DCM and stirred at room temperature (approximately 25° C.) for 24 hours. The DCM was then removed by slow evaporation at room temperature. The observed precipitate was isolated by centrifugation and dried at 40° C. under vacuum for overnight. Melting point=195-197° C. (onset).

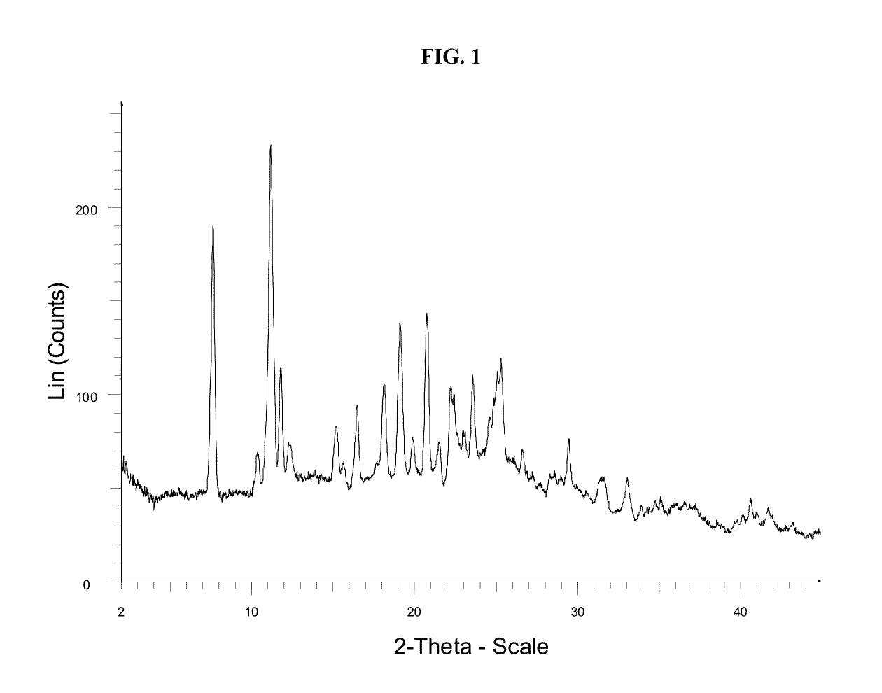

The powder X-ray diffraction pattern for “Form I” is shown in FIG. 1 and the differential scanning calorimetry (DSC) and thermogravimetric analysis (TGA) thermograms are shown in FIG. 2 and FIG. 3, respectively. Form I is an ansolvated form.

The X-ray diffraction pattern for the “Form I” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate shown in FIG. 1 was generated using the following settings:

Type: 2 Theta alone

Start: 3.000°

End: 45.000°

Step: 0.020°

Step time: 120 seconds

Temperature: Room temperature (approximate 25° C.)

Time Started: 0 seconds

X: 44.75 mm

Y: −34.11 mm

Z: 39.47 mm

### Preparation of “Form II” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

5.0 mg of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate obtained according to Example 1, was suspended in 100 μL of acetone. The suspension was heated to 55° C. at a rate of 0.5° C./min. A clear solution was obtained upon heating. The solution was then cooled down to 5° C. at a rate of 0.25° C./min. The heating-cooling process was repeated for 6 cycles. The precipitation was isolated by centrifugation and dried at 40° C. under vacuum for overnight.

Alternatively, the “Form II” crystalline form may be prepared by substituting acetone with ACN or 2-propanol.

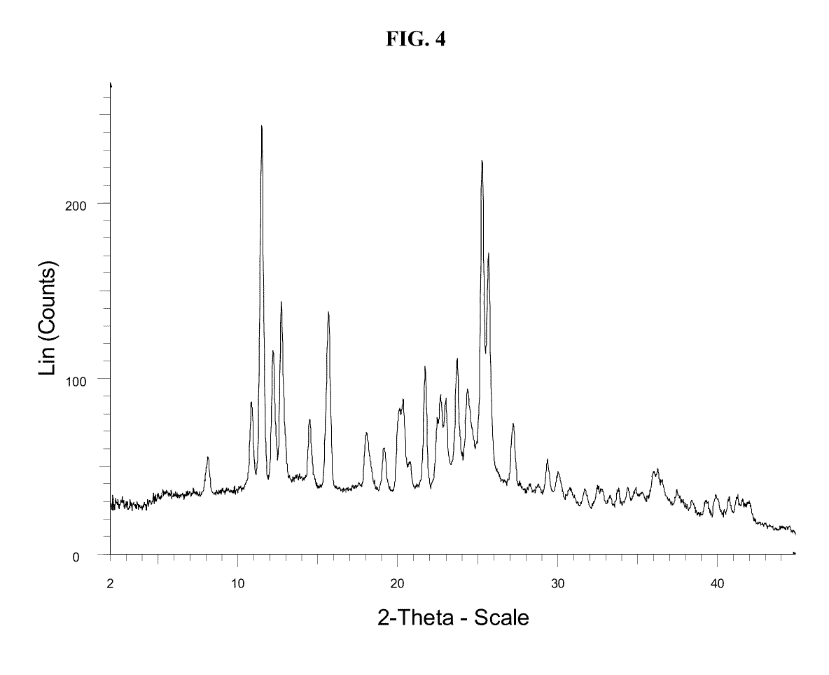

Melting point=218-220° C. (onset). The powder X-ray diffraction pattern is shown in FIG. 4 and the differential scanning calorimetry (DSC) thermogram and thermogravimetric analysis (TGA) thermograms are shown in FIG. 5 and FIG. 6, respectively. Form II is an ansolvated form. Since “Form II” is found to be the prevailing form in a series of crystallization experiments, it is considered the more thermodynamically stable form.

The X-ray diffraction pattern for the “Form II” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate shown in FIG. 4 was generated using the following settings:

Type: 2 Theta alone

Start: 2.000°

End: 45.000°

Step: 0.020°

Step time: 120 seconds

Temperature: Room temperature (approximate 25° C.)

Time Started: 0 seconds

X: 34.45 mm

Y: −1.55 mm

Z: 39.43 mm

### Preparation of “Form III” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate

5.0 mg of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate obtained according to Example 1, was suspended in 100 μL of EA. The suspension was heated to 55° C. at a rate of 0.5° C./min. A clear solution was obtained upon heating. The solution was then cooled down to 5° C. at a rate of 0.25° C./min. The heating-cooling process was repeated for 6 cycles. The precipitation was isolated by centrifugation and dried at 40° C. under vacuum for overnight.

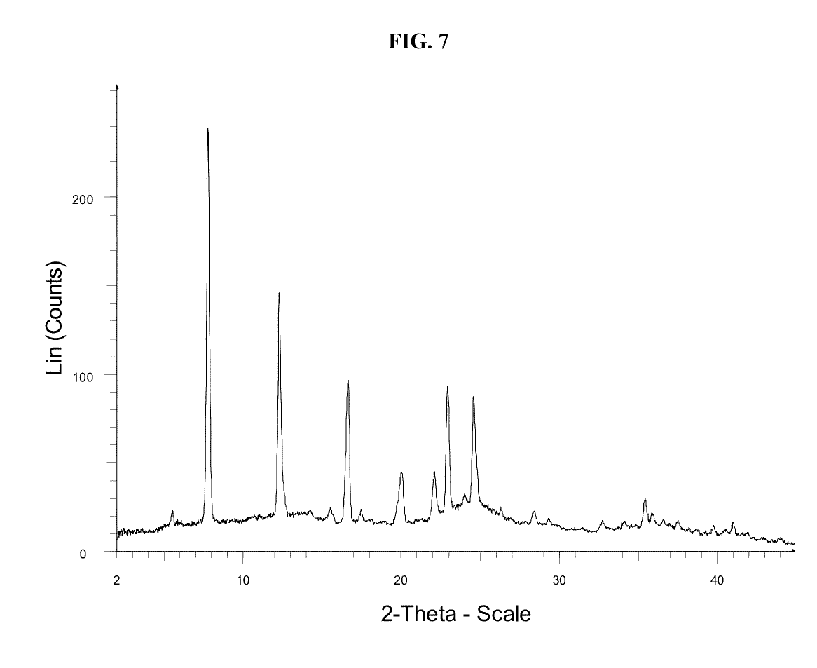

The powder X-ray diffraction pattern is shown in FIG. 7 and the differential scanning calorimetry (DSC) thermogram is shown in FIG. 8.

The X-ray diffraction pattern for the “Form III” crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate shown in FIG. 7 was generated using the following settings:

Type: 2 Theta alone

Start: 3.000°

End: 45.000°

Step: 0.020°

Step time: 120 seconds

Temperature: Room temperature (approximate 25° C.)

Time Started: 0 seconds

X: 29.23 mm

Y: −25.23 mm

Z: 39.46 mm

### Example 19

### Solubility of Form I and II from Examples 16 and 17 Respectively

Form I crystalline form and Form II crystalline form are chemically and physically stable in bulk state for one week when stored at 50° C., 50° C./75% RH and 80° C. Form III crystalline form is a metastable form and converts to Form II crystalline form after being heated at approximate 142° C. Form II crystalline form has lower solubility compared to Form I crystalline form indicating that Form II crystalline form is thermodynamically more stable than Form I crystalline form, See below Table 11.

TABLE 11

| | Solubility | Solubility | |
|---|---|---|---|
| | of Form I, | of Form II, | |
| Media | mg/ml [pH] | mg/ml [pH] | |
| pH 1 (0.1N HCl) | 1.249 [1.39] | 0.246 | [1.39] |
| pH 3 citrate buffer | 0.816 [3.04] | 0.0740 | [2.96[ |
| pH 4.65 acetate buffer | 0.687 [4.67] | 0.0557 | [4.67] |
| pH 6.8 phosphate buffer | 0.794 [6.70] | 0.0551 | [6.47] |
| Water | 0.799 [3.82] | 0.0676 | [5.97] |
| Simulated gastric fluid | 0.681 [2.11] | 0.150 | [2.15] |
| [pH 2] | | | |
| Fasted state simulated | 0.632 [6.36] | 0.0888 | [6.30] |
| intestinal fluid [pH 6.5] | | | |
| Fed state simulated | 0.665 [5.80] | 0.0995 | [5.76] |
| intestinal fluid [pH 5.8] | | | |

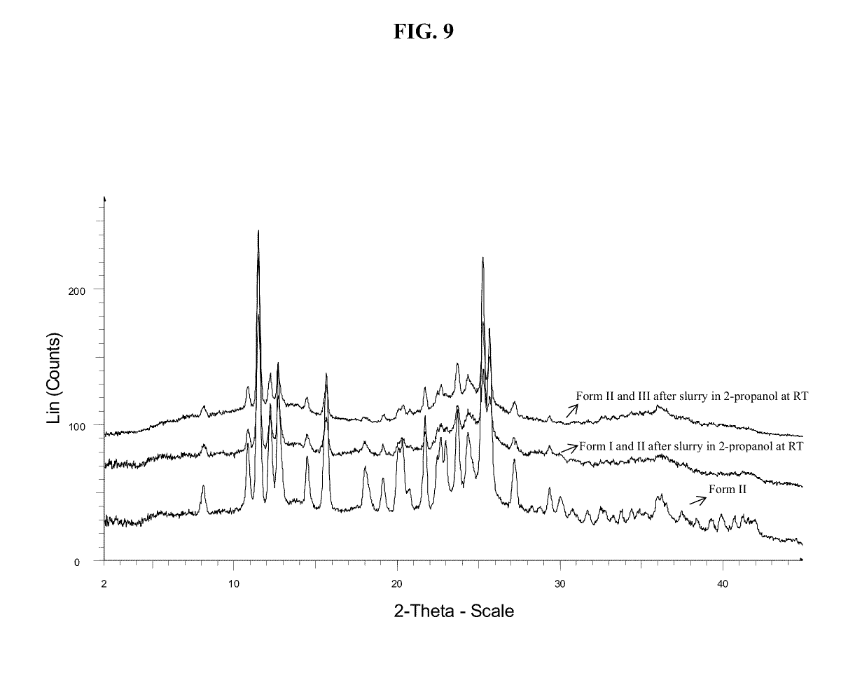

In addition, Slurry competition results also suggest that Form II is thermodynamically more stable than Form I and III, See FIG. 9.

What is claimed is:

1. A compound of Formula (I) 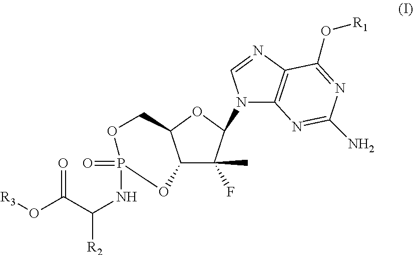
wherein
R₁ is methyl, ethyl, n-propyl or i-propyl;
R₂ is H, methyl, ethyl, n-propyl or i-propyl; and
R₃ is methyl, ethyl, n-propyl or i-propyl.

2. The compound of claim 1 having a structure of Formula (II)

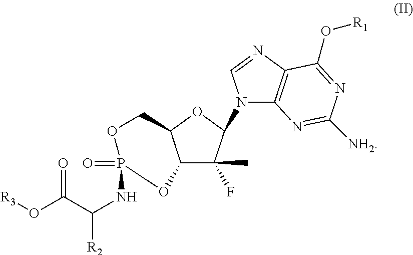

3. The compound of claim 1 having a structure of Formula (III)

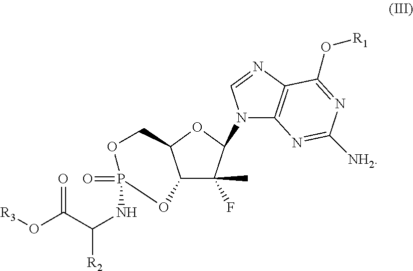

4. The compound of claim 1 having a structure of Formula (IV)

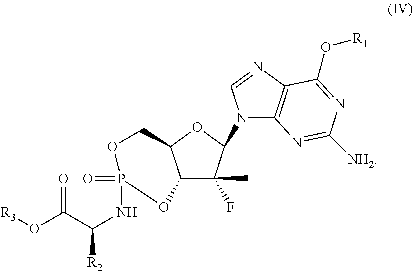

5. The compound of claim 1 having a structure of Formula (V)

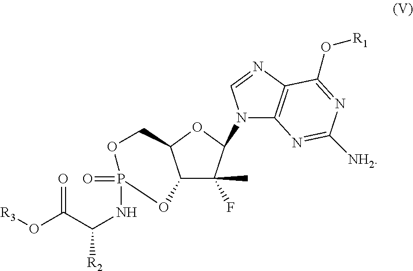

6. The compound of claim 1, wherein R₁ is ethyl, n-propyl or i-propyl.

7. The compound of claim 1, wherein R₂ is H, methyl or i-propyl.

8. The compound of claim 1, wherein R₃ is methyl, ethyl or i-propyl.

9. The compound of claim 1, selected from the group consisting of:

| | |
|---|---|
| 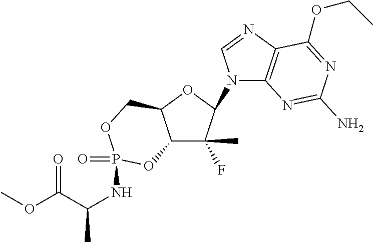 [SMILES: CCOc1nc(N)nc2c1ncn2[C@@H]1O[C@@H]2CO[P@](=O)(N[C@@H](C)C(=O)OC)O[C@H]2[C@@]1(C)F] | (S)-methyl 2- (((2R,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 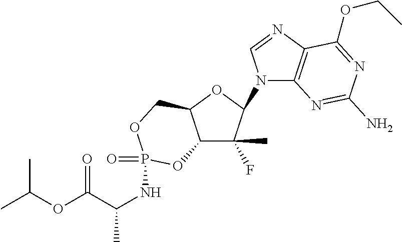 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | (R)-isopropyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 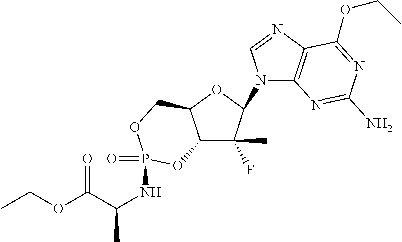 [SMILES: CCOC(=O)[C@H](C)N[P@@]1(=O)OC[C@H]2O[C@@H](n3cnc4c(OCC)nc(N)nc43)[C@](C)(F)[C@@H]2O1] | (S)-ethyl 2- (((2R,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 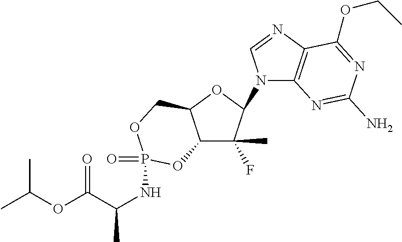 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F] | (S)-isopropyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 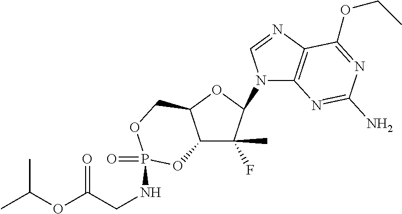 [SMILES: CCOc1nc(N)nc2c1ncn2[C@@H]1O[C@@H]2CO[P@](=O)(NCC(=O)OC(C)C)O[C@H]2[C@@]1(C)F] | isopropyl 2- (((2R,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)acetate, |
| 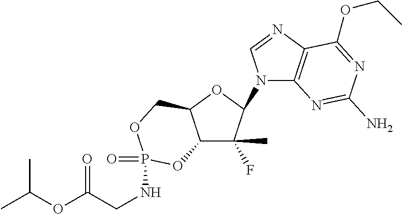 [SMILES: CCOc1nc(N)nc2c1ncn2[C@@H]1O[C@@H]2CO[P@@](=O)(NCC(=O)OC(C)C)O[C@H]2[C@@]1(C)F] | isopropyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)acetate, |
| 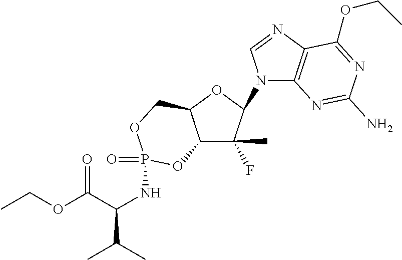 [SMILES: CCOC(=O)[C@@H](N[P@]1(=O)OC[C@H]2O[C@@H](n3cnc4c(OCC)nc(N)nc43)[C@](C)(F)[C@@H]2O1)C(C)C] | (S)-ethyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)-3-methylbutanoate, |
| 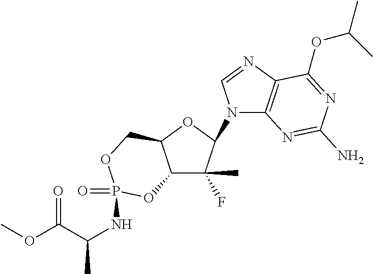 [SMILES: COC(=O)[C@H](C)N[P@@]1(=O)OC[C@H]2O[C@@H](n3cnc4c(OC(C)C)nc(N)nc43)[C@](C)(F)[C@@H]2O1] | (S)-methyl 2- (((2R,4aR,6R,7R,7aR)-6-(2- amino-6-isopropoxy-9H- purin-9-yl)-7-fluoro-7-methyl- 2-oxidotetrahydro-4H- furo[3,2- d][1,3,2]dioxaphosphinin-2- yl)amino)propanoate, and |
| 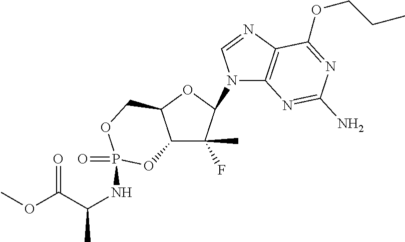 [SMILES: CCCOc1nc(N)nc2c1ncn2[C@@H]1O[C@@H]2CO[P@](=O)(N[C@@H](C)C(=O)OC)O[C@H]2[C@@]1(C)F] | (S)-methyl 2- (((2R,4aR,6R,7R,7aR)-6-(2- amino-6-propoxy-9H-purin-9- yl)-7-fluoro-7-methyl-2- oxidotetrahydro-4H-furo[3,2- d][1,3,2]dioxaphosphinin-2- yl)amino)propanoate. |

10. The compound of claim 1, selected from the group consisting of:

| | |
|---|---|
| 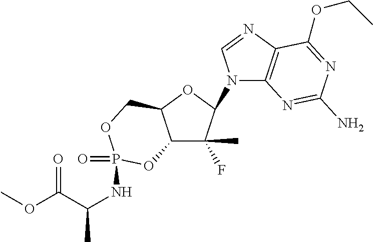 [SMILES: CCOc1nc(N)nc2c1ncn2[C@@H]1O[C@@H]2CO[P@](=O)(N[C@@H](C)C(=O)OC)O[C@H]2[C@@]1(C)F] | (S)-methyl 2- (((2R,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 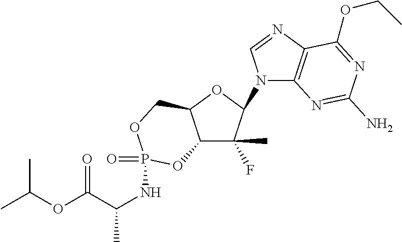 [SMILES: CCOc1nc(N)nc2c1ncn2[C@@H]1O[C@@H]2CO[P@@](=O)(N[C@H](C)C(=O)OC(C)C)O[C@H]2[C@@]1(C)F] | (R)-isopropyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, |
| 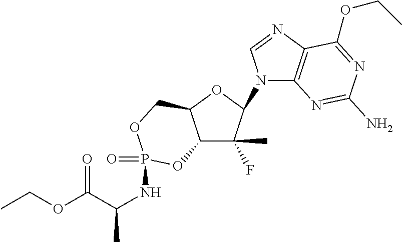 [SMILES: CCOC(=O)[C@H](C)N[P@@]1(=O)OC[C@H]2O[C@@H](n3cnc4c(OCC)nc(N)nc43)[C@](C)(F)[C@@H]2O1] | (S)-ethyl 2- (((2R,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate, and |
| 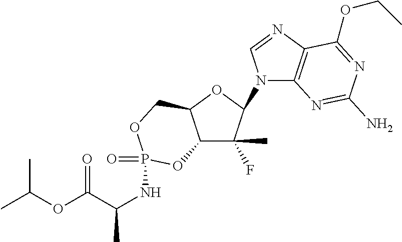 [SMILES: CCOc1nc(N)nc2c1ncn2[C@@H]1O[C@@H]2CO[P@@](=O)(N[C@@H](C)C(=O)OC(C)C)O[C@H]2[C@@]1(C)F] | (S)-isopropyl 2- (((2S,4aR,6R,7R,7aR)-6-(2- amino-6-ethoxy-9H-purin-9- yl)-7-fluoro-7-methyl-2- oxidotetrahydro-4H-furo[3,2- d][1,3,2]dioxaphosphinin-2- yl)amino)propanoate. |

11. The compound of claim 1, having the structure

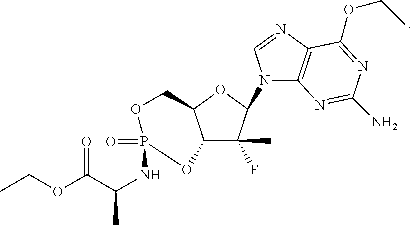 [SMILES: CCOC(=O)[C@H](C)N[P@@]1(=O)OC[C@H]2O[C@@H](n3cnc4c(OCC)nc(N)nc43)[C@](C)(F)[C@@H]2O1]

12. A pharmaceutical composition comprising a therapeutically effective amount of a compound according to any one of the claims 1-11, and a pharmaceutically acceptable carrier or excipient.

13. The pharmaceutical composition of claim 12 further comprising at least one additional pharmaceutical agent.

14. The pharmaceutical composition of claim 13 wherein said at least one additional pharmaceutical agent is selected from the group consisting of interferons, ribavirin and ribavirin analogs, cyclophilin binder, HCV NS3 protease inhibitors, HCV NS5a inhibitors, P7 inhibitor, entry inhibitor, NS4b inhibitor, alpha-glucosidase inhibitors, host protease inhibitors, immune modulators, symptomatic relief agents, nucleoside and non-nucleoside NS5b inhibitors.

15. A method for treating a disease caused by a viral infection comprising the step of administering to a subject in need thereof a therapeutically effective amount of a compound according to any one of the claims 1-11.

16. The method of claim 15 wherein said subject is human.

17. The method of claim 15 wherein said viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

18. The method of claim 15 wherein said viral infection is caused by dengue virus.

19. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 1.

20. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a powder X-ray diffraction pattern comprising peaks at diffraction angles (2θ) of 7.6°, 10.3°, 11.1°, 11.8°, 12.3°, 15.2°, 16.5°, 18.1°, 19.9°, 20.7°, 21.5°, 22.2°, 23.6°, 25.3°, 25.7° and 29.5°.

21. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 4.

22. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a powder X-ray diffraction pattern comprising peaks at diffraction angles (2θ) of 8.1°, 10.8°, 11.4°, 12.2°, 12.7°, 14.5°, 15.6°, 18.1°, 19.1°, 20.1°, 20.3°, 21.7°, 22.7°, 23.0°, 23.7°, 24.4°, 25.3°, 25.7° and 27.2°.

23. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a X-ray diffraction spectrum substantially the same as the X-ray powder diffraction spectrum shown in FIG. 7.

24. A crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate having a powder X-ray diffraction pattern comprising peaks at diffraction angles (2θ) of 7.7°, 12.3°, 15.5°, 16.6°, 17.4°, 20.0°, 22.1°, 22.9°, 24.6° and 35.6°.

25. The crystalline form of any one of the claims 19-24 wherein said crystalline form is substantially pure.

26. A pharmaceutical composition comprising a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate in accordance with any one of the claims 19-24; and a pharmaceutically acceptable excipient, diluent or carrier.

27. The pharmaceutical composition of claim 26 further comprising at least one additional pharmaceutical agent.

28. The pharmaceutical composition of claim 27 wherein said at least one additional pharmaceutical agent is selected from the group consisting of interferons, ribavirin and ribavirin analogs, cyclophilin binder, HCV NS3 protease inhibitors, HCV NS5a inhibitors, P7 inhibitor, entry inhibitor, NS4b inhibitor, alpha-glucosidase inhibitors, host protease inhibitors, immune modulators, symptomatic relief agents, nucleoside and non-nucleoside NS5b inhibitors.

29. A method of treating a disease caused by a viral infection comprising the step of administering to a mammal in need thereof a therapeutically effective amount of a crystalline form of (S)-ethyl 2-(((2R,4aR,6R,7R,7aR)-6-(2-amino-6-ethoxy-9H-purin-9-yl)-7-fluoro-7-methyl-2-oxidotetrahydro-4H-furo[3,2-d][1,3,2]dioxaphosphinin-2-yl)amino)propanoate in accordance with any one of the claims 19-24, or a pharmaceutical composition thereof.

30. The method of claim 29 wherein said viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

31. The method of claim 29 wherein said viral infection is caused by dengue virus.
